(19) United States
(12) Patent Application Publication
(10) Pub. No.: US 2021/0388007 A1
(43) Pub. Date: Dec. 16, 2021

(54) COMPOUND AND PREPARATION METHOD AND APPLICATION THEREOF

(71) Applicant: WUHAN UNIVERSITY, HUBEI (CN)

(72) Inventors: Xiao SHEN, Hubei (CN); Xiang CHEN, Hubei (CN); Xingxing GONG, Hubei (CN)

(73) Assignee: WUHAN UNIVERSITY, HUBEI (CN)

(21) Appl. No.: 17/336,330

(22) Filed: Jun. 2, 2021

(30) Foreign Application Priority Data
Jun. 11, 2020 (CN) 202010529282.X

(51) Int. Cl. C07F 7/08; C07C 67/31; C07D 209/34; C07C 45/64; C07D 307/46; C07C 231/12; C07C 253/30; C07C 29/32; C07J 1/00; C07D 317/54; C07C 315/04; C07J 71/00

(52) U.S. Cl. CPC C07F 7/081; C07F 7/0827; C07F 7/083; C07C 67/31; C07D 209/34; C07C 45/64; C07J 71/0031; C07C 231/12; C07C 253/30; C07C 29/32; C07J 1/0011; C07D 317/54; C07C 315/04; C07D 307/46

(57) ABSTRACT

The present disclosure relates to a compound and a preparation method and application thereof, the compound having a chemical structure formula of:

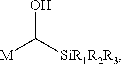

wherein M in the formula is selected from a group consisting of CF₃ or CF₂H, and R₁, R₂, and R₃ are each independently selected from a group consisting of aryl, heteroaryl, and alkyl. The compound provided by the present disclosure can be used as a trifluoroethanolation reagent or difluoroethanolation reagent as synthetic intermediates of many organic compounds, and some of the compounds have pharmaceutical activity. The preparation steps of such compounds are simplified, with mild synthesis conditions and wide applicability of substrates.

### CROSS-REFERENCE TO RELATED APPLICATION

This application claims the priority benefit of China application no. 202010529282.X, filed on Jun. 11, 2020. The entirety of the above mentioned patent application is hereby incorporated by reference herein and made a part of this specification.

### FIELD

The present disclosure belongs to the technical field of organic synthesis and relates to a compound and a preparation method and application thereof.

### BACKGROUND

The introduction of fluorine atoms into compounds has become a common strategy in the synthesis of medications or the like, and the introduction of fluorine atoms can enhance or change the chemical, physical and biological properties of organic compounds [a) P. Kirsch, Modern Fluoroorganic Chemistry: Synthesis, Reactivity, Applications, Wiley-VCH, 2013; b) J.-P. Bégué, D. Bonnet-Delpon, Bioorganic and Medicinal Chemistry Wiley-VCH, Weinheim, 2008; c) C. Ni, M. Hu, J. Hu, Chem. Rev. 2015, 115, 765; d) Liang, T.; Neumann, C. N.; Ritter, T. Angew. Chem., Int. Ed. 2013, 52, 8214; Angew. Chem. 2013, 125, 8372.]. Common fluoroalkyl silicon reagents, for example, Rupert-Prakash reagent and TMSCF₂H, are widely used in the synthesis of organo-fluorine compounds. However, such classical method for obtaining a target product by nucleophilic addition of an aldehyde compound and a carbanion intermediate formed with anion-activated C—Si still has some shortcomings: 1) regioselectivity in compounds containing multiple aldehyde groups is difficult to control; 2) many aldehyde compounds, particularly alkyl aldehyde, are unstable and the synthesis of specific aldehydes requires multiple steps [a) Y. He, M.-M. Tian, X.-Y. Zhang, X.-S. Fan, Asian J. Org. Chem. 2016, 5, 1318; b) W. Ou, G. Zhang, J. Wu, C. Su, ACS Cat. 2019, 9, 5178-5183.].

In order to solve the above problems, the applicant develops a class of compounds that can be used as trifluoroethanolation reagents (A) or difluoroethanolation reagents (B), which are chemically stable and have good compatibility upon participation in reaction, and can be applied in the late-course of a functionalization reaction of drug molecules or other functional molecules, and fluoroethanol-containing α-allylation, alkylation, alkenylation reactions can be achieved by using the radical substitution/addition method to obtain a series of tri-/difluoromethyl-substituted homoallyl alcohol compounds, alkyl-substituted tri-/difluoroethanol compounds and tri-/difluoromethyl-substituted allyl alcohol compounds.

### SUMMARY

The technical problem to be solved by the present disclosure is to provide a novel compound and a preparation method and application thereof aiming at the above deficiencies existing in the prior art. The compound may be used as a fluorine-containing fluoroethanolation with easily available raw material, simple preparation process, mild conditions, and a wide range of substrate applicability, and a very efficient and convergent method may be provided for synthesizing products such as drug molecules containing trifluoroethanol structural or difluoroethanol structural units.

To solve the above technical problem, the present disclosure provides the following technical solutions:

A compound, having a chemical structure formula of:

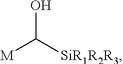

wherein M is selected from a group consisting of CF₃ or CF₂H, and R₁, R₂, and R₃ are each independently selected from a group consisting of aryl, heteroaryl, and alkyl.

The aryl as described in the present disclosure refers to an optionally substituted aromatic hydrocarbon group having 6 to about 20, for example 6 to 12 or 6 to 10 ring-forming carbon atoms, which may be monocyclic aryl, bicyclic aryl, or polycyclic aryl. The bicyclic aryl group or the polycyclic aryl may be a structure of a monocyclic aryl group fused with other independent rings, such as an alicyclic, heterocyclic, aromatic, or aromatic heterocyclic ring.

The aryl as described in the present disclosure may have one or more substituents, and the substituents are not limited in any way. Common substituents include such as aryl, alkyl, an ester group, cyano, nitro, an amide group, sulfonyl, alkoxy, alkenyl, alkynyl, an aldehyde group, hydroxyl and halogen. The aromatic group may have one or more of these substituents thereon, and when multiple substituents are present, the multiple substituents may be the same or different.

The heteroaryl as described in the present disclosure refers to an optionally substituted heteroaryl group including from about 5 to about 20, such as 5 to 12, or 5 to 10 skeleton ring-forming atoms, wherein at least one of the ring-forming atoms is a heteroatom independently selected from a group consisting of oxygen, nitrogen, sulfur, phosphorus, silicon, selenium and tin, but is not limited thereto. The ring of said group does not contain two adjacent O or S atoms. The heteroaryl includes monocyclic heteroaryl (having one ring), bicyclic heteroaryl (having two rings), or polycyclic heteroaryl (having two or more rings). In embodiments where two or more heteroatoms are present in the ring, the two or more heteroatoms may be identical to each other, or some or all of the two or more heteroatoms may be different from each other. The bicyclic heteroaryl or the polycyclic heteroaryl may be a structure of a monocyclic heteroaryl fused with other independent rings, such as an alicyclic, heterocyclic, aromatic, or aromatic heterocyclic ring (which may be collectively referred to as a fused cyclic heteroaryl). Non-limiting examples of the monocyclic heteroaryl include monocyclic heteroaryl with 5 to about 12, 5 to about 10, 5 to about 7, or 6 skeleton ring-forming atoms, for example, including pyridyl; the fused cyclic heteroaryl includes benzimidazolyl, quinolinyl, and acridinyl. Other examples of the heteroaryl include, but are not limited to: pyridyl, pyrimidinyl, pyrazinyl, pyridazinyl, triazinyl, furanyl, thienyl, imidazolyl, triazolyl, tetrazolyl, thiazolyl, isothiazolyl, 1,2,4-thiadiazolyl, pyrrolyl, pyrazolyl, oxazolyl, isoxazolyl, oxadiazolyl, benzofuranyl, benzothienyl, benzothiazolyl, indolyl, indazolyl, quinolinyl, isoquinolinyl, purinyl, carbazolyl, benzimidazolyl, pyrrolopyridyl, pyrrolopyrimidinyl, pyrazolopyridyl, pyrazolopyrimidinyl, acridinyl, phenazinyl, benzoxazolyl, benzothiadiazolyl, benzoxadiazolyl, benzotriazolyl, isoquinolinyl, indyl, isothiazolyl, pseudoindolyl, oxadiazolyl, purinyl, phthalazinyl, pteridyl, quinazolinyl, quinoxalinyl, triazinyl and thiadiazolyl, and so on, and oxides thereof, such as pyridinyl-N-oxide.

The alkyl as described in the present disclosure refers to an optionally substituted saturated aliphatic hydrocarbon, which is a linear, cyclic or branched structure, preferably having from 1 to about 20 carbon atoms, for example having from 1 to about 10 carbon atoms, having from 1 to about 8 carbon atoms, or from 1 to about 6 carbon atoms, or from 1 to about 4 carbon atoms or from 1 to about 3 carbon atoms. Examples of alkyl of the present disclosure include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, tert-butyl, n-pentyl, iso-pentyl, neopentyl, tert-pentyl and hexyl, and longer alkyl groups, such as heptyl and octyl. These alkyl groups may contain one or more substituents, which may be, but not limited to, aryl, alkyl, aroyl, alkanoyl, substituted oxyacyl, alkoxy, halogen, alkoxy, etc., alkenyl, alkynyl, heteroaryl. The substituents may be at different positions of the alkyl group. One or more substituents may be present, and where it may be substituted by multiple functional groups, the functional groups may have identical or different types, and may be at the same or different positions.

Preferably, the compound is one of the following substances:

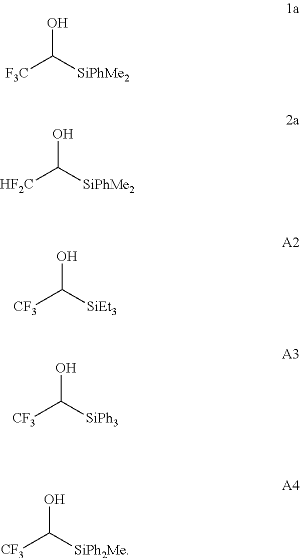

The present disclosure further includes a method for preparing the above compound.

When M is CF₃, the method specifically includes the following steps:

1) adding R₁, R₂, R₃-trisubstituted chlorosilane (ClSiR₁R₂R₃), trifluoroethanol (3) and hexamethylphosphoramide (HMPA) to a solvent (tetrahydrofuran, THF), performing cooling to −80° C. to −60° C., and then adding dropwise lithium diisopropylamide (LDA) with a syringe pump; after completion of adding dropwise, performing thorough stirring, then performing heating to room temperature, and performing stirring again until trifluoroethanol is completely consumed; adding triethylchlorosilane (ClSiEt₃) at 0-4° C., performing stirring for a certain time, and performing separating by column chromatography to obtain a compound 4;

2) adding a fluorinating agent (preferably Select-Fluor) to a mixed solvent of acetonitrile and dichloromethane (DCM) (a volume ratio of acetonitrile to DCM is 4:1), adding the compound 4 at 0-4° C., subsequently carrying out a reaction at room temperature for 8-12 h, quenching the reaction with water, and then performing separating by column chromatography to obtain a compound 5; and

(3) adding the compound 5 to methanol (MeOH), followed by addition of sodium borohydride (NaBH₄) in batches, and carrying out a reaction at 0-4° C.; after completion of the reaction, adding water to quench the reaction, and performing separating by column chromatography to obtain a compound A;

wherein, the chemical equation is as follows:

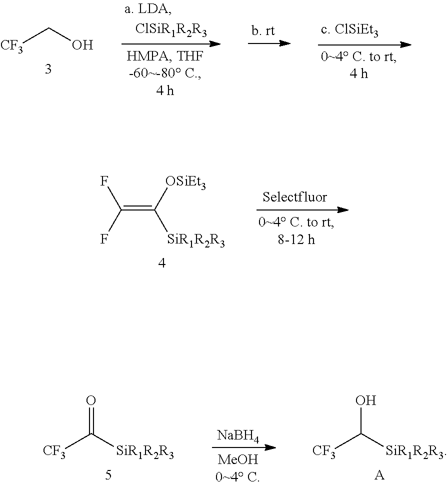

According to the above solution, a molar ratio of R₁, R₂, R₃-trisubstituted chlorosilane to trifluoroethanol to LDA in the step 1) is 1:1:3.5.

According to the above solution, a molar ratio of the fluorinating agent to the compound 4 in the step 2) is 2:1.

According to the above solution, a molar ratio of the total addition amount of sodium borohydride to the compound 5 in the step 3) is 1:1.

When M is CF₂H, the method specifically includes the following steps:

1) adding a compound 4 into a solvent (THF), and adding dropwise concentrated hydrochloric acid to the resulting solution at 0˜4° C. After completion of adding dropwise, performing heating to room temperature and carrying out a reaction under stirring; after completion of the reaction, adding water to quench the reaction and performing separating by column chromatography to obtain a compound 6; and

(2) dissolving the compound 6 in a solvent (methanol), adding sodium borohydride in batches at 0-4° C., and carrying out a reaction; after completion of the reaction, adding water to quench the reaction, and performing separating by column chromatography to obtain a compound B;

wherein, the chemical equation is as follows:

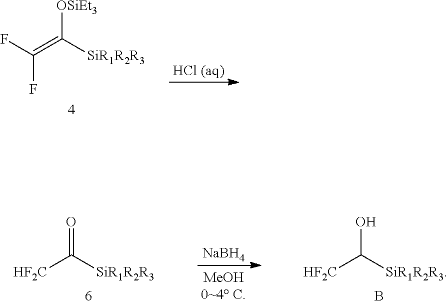

According to the above solution, a molar ratio of the compound 4 to HCl in concentrated hydrochloric acid in step 1) is 1:10.

According to the above solution, a molar ratio of sodium borohydride to a compound 6 in the step 2) is 1.1:1, with sodium borohydride added in three batches.

The present disclosure further includes an application of the above compound, wherein the compound is used as a trifluoroethanolation reagent when M is CF₃, and the compound is used as a difluoroethanolation reagent when M is CF₂H.

Examples of the specific applications are as follows:

I. A trifluoromethyl homoallyl alcohol compound may be prepared according to allylation of the above trifluoroethanolation reagent (A), specifically by the steps of: carrying out a reaction between the trifluoroethanolation reagent, allyl sulfone C, a catalyst D, and an oxidant E under stirring in an organic solvent F at 50-100° C. under nitrogen protection; after completion of the reaction, quenching the reaction by a tetrabutylammonium fluoride (TBAF) solution, and then performing separating and purifying to obtain the corresponding α-trifluoromethyl homoallyl alcohol compound (G).

The reaction formula for the method of the present disclosure may be expressed as follows:

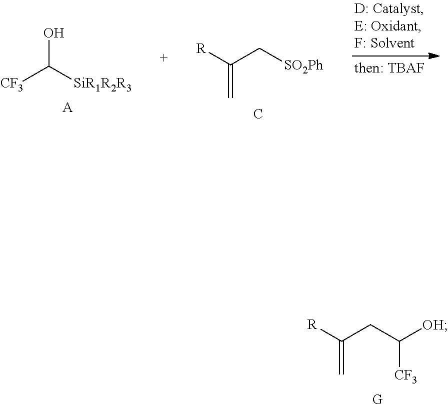

wherein the R group represents a substituent group on allyl sulfone, and may be aryl, heteroaryl, alkyl, substituted alkyl (including an alkyl group substituted by an oxygen atom group, and a nitrogen atom group), aroyl, alkanoyl, substituted oxyacyl, substituted aminoacyl, halogen, sulfonyl, substituted sulfuryl, alkenyl, alkynyl, cyano, nitro, amido, an aldehyde group, and so on.

The catalyst D preferably employs a manganese catalyst to promote the reaction, and the available manganese catalysts include divalent or trivalent manganese compounds, such as manganese (III) acetate dihydrate, manganese (II) acetate tetrahydrate, manganese (III) phosphate, and manganese (III) acetylacetonate.

The oxidant E is preferably a peroxide to promote the reaction, and the available peroxides include TBHP (tert-butyl hydroperoxide), DCP (dicumyl peroxide), DTBP (di-tert-butyl peroxide), TBPB (tert-butyl perbenzoate), and so on.

The solvent F is a conventional solvent, and selected from a group consisting of methanol, ethanol, isopropanol, tert-butanol, tetrahydrofuran, 2-methyltetrahydrofuran, diethyl ether, dimethyl ethylene glycol ether, methyl tert-butyl ether, 1,4-dioxane, 1,3-dioxane, dichloromethane, 1,2-dichloroethane, chloroform, carbon tetrachloride, saturated C4-12 alkane, fluorinated or chlorinated C3-12 alkane, benzene, toluene, xylene, trimethylbenzene, dimethyl sulfoxide, N,N-dimethylformamide (DMF), N,N-dimethylacetamide, acetone, N-methylpyrrolidone, acetonitrile, saturated C3-12 alkyl nitrile, and so on.

The preferred reaction condition: the reaction is carried out at 50-100° C.

According to the above solution, the aroyl group refers to a functional group having the following structure:

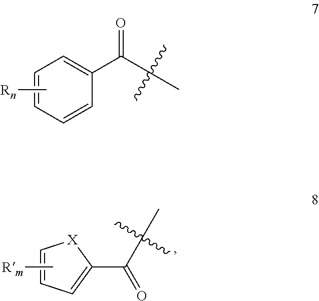

wherein, in the functional group 7, R represents a substituent group on an aryl group, and the letter n represents the number of substituent groups on the aryl group, 0≤n≤4. The functional group is not limited in any way, and the common substituent group includes aryl, alkyl, an ester group, cyano, nitro, an amide group, sulfonyl, alkoxy, halogen and so on, and when multiple substituent groups are contained, these functional groups may be the same or different. In the functional group 8, X denotes a heteroatom or a heteroatom group, such as N, S, O, for forming a cyclic aryl structure, R′ represents a substituent group on a heterocyclic aromatic ring, and the letter m represents the number of substituent groups, 0≤m≤3, the functional group is not limited in any way, and the common substituent group includes aryl, alkyl, an ester group, cyano, nitro, an amide group, sulfonyl, alkoxy, halogen and so on, and when multiple substituent groups are contained, these functional groups may be the same or different.

According to the above solution, the alkanoyl is a functional group having the following structure:

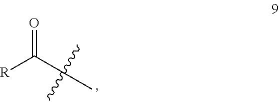

wherein, R represents an alkyl group.

According to the above solution, the substituted oxyacyl is a functional group having the following structure:

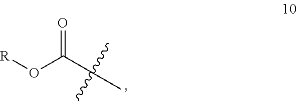

wherein R denotes a functional group substituted on oxygen, and is selected from a group consisting of alkyl, aryl, and a cutting moiety of a natural organic compound, wherein the cutting moiety of the natural organic compound is a natural organic compound containing hydroxyl groups, including menthol, cholesterol, testosterone, diosgenin, epiandrosterone, vitamin E, estradiol, and so on.

According to the above solution, the substituted aminoacyl refers to a functional group having the following structure:

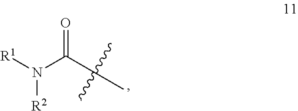

wherein R¹ and R² are each independently selected from a group consisting of aryl or alkyl. The two substituents may be the same or different.

II. A trifluoromethyl alkyl alcohol compound may be prepared according to an alkylation reaction of the above trifluoroethanolation reagent (A) with acrylamide, specifically by the steps of: carrying out a reaction between the trifluoroethanolation reagent, acrylamide H, a catalyst D, and an oxidant E under stirring in an organic solvent F at 50-100° C. under nitrogen protection; after completion of the reaction, quenching the reaction by a tetrabutylammonium fluoride solution, and then performing separating and purifying to obtain the corresponding α-trifluoromethyl alkyl alcohol compound I.

The reaction formula for the method of the present disclosure may be expressed as follows:

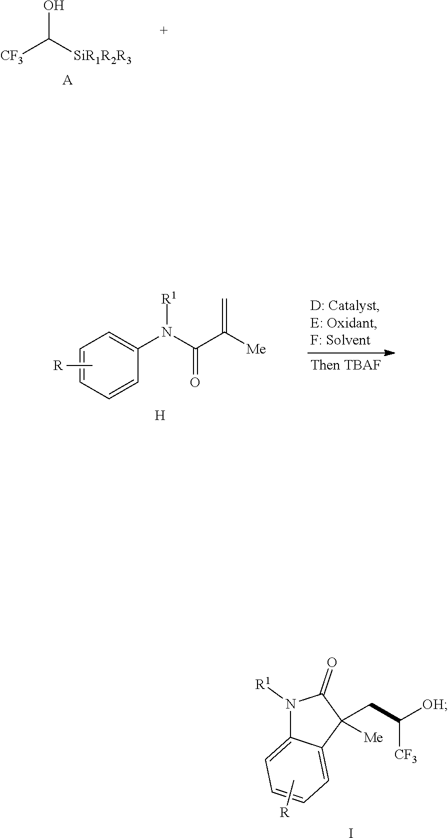

wherein the R group represents a substituent group on the aromatic ring of acrylamide, and includes aryl, alkyl, aroyl, alkanoyl, substituted oxyacyl, halogen, cyano, nitro, alkoxy and so on. The substituent group may be one or more, and when multiple substituent groups are contained, these substituent groups may be the same or different. R¹ represents another substituent group on the nitrogen atom, and may be aryl, alkyl, aroyl, alkanoyl, and so on.

III. A trifluoromethyl allyl alcohol compound may be prepared according to an alkenylation reaction of the above trifluoroethanolation reagent (A) with cinnamic acid, specifically by the steps of: carrying out a reaction between the trifluoroethanolation reagent A, cinnamic acid J, a catalyst D, and an oxidant E under stirring in an organic solvent F at 50-100° C. under nitrogen protection; after completion of the reaction, quenching the reaction by a tetrabutylammonium fluoride solution, and then performing separating and purifying to obtain the corresponding α-trifluoromethyl allyl alcohol compound K.

The reaction formula for the method of the present disclosure may be expressed as follows:

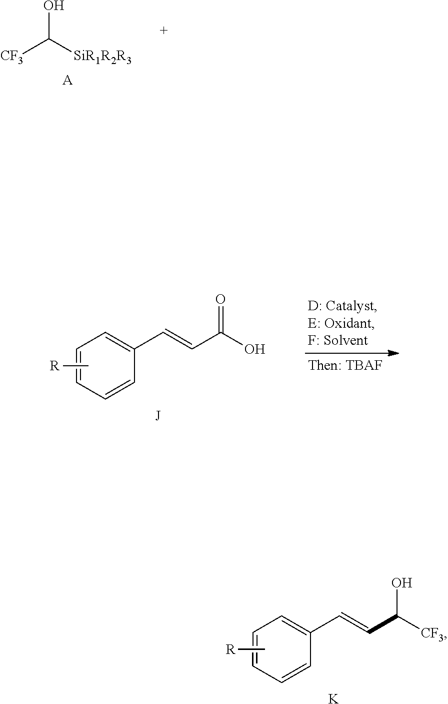

wherein the R group represents a substituent group on the aromatic ring of cinnamic acid, and may be aryl, alkyl, aroyl, alkanoyl, substituted oxyacyl, halogen, alkoxy and so on. The substituent group may be one or more, and when a plurality of substituent groups are contained, these substituent groups may be the same or different.

IV. A difluoromethyl homoallyl alcohol compound G may be prepared according to an reaction of the above difluoroethanolation reagent (B) with allyl sulfone C, specifically by the steps of: carrying out a reaction between the difluoroethanolation reagent B, allyl sulfone C, a catalyst D, and an oxidant E under stirring in an organic solvent F at 50-100° C.; after completion of the reaction, quenching the reaction by a tetrabutylammonium fluoride solution, and then performing separating and purifying to obtain the α-difluoromethyl homoallyl alcohol compound G.

V. A difluoromethyl alkyl alcohol compound I may be prepared according to an reaction of the above difluoroethanolation reagent (B) with acrylamide H, specifically by the steps of: carrying out a reaction between the difluoroethanolation reagent B, acrylamide H, a catalyst D, and an oxidant E under stirring in an organic solvent F at 50-100° C. under nitrogen protection; after completion of the reaction, quenching the reaction by a tetrabutylammonium fluoride solution and then performing separating and purifying to obtain the α-difluoromethyl alkyl alcohol compound I.

VI. A difluoromethyl allyl alcohol compound K may be prepared according to an reaction of the above difluoroethanolation reagent (B) with cinnamic acid J, specifically by the steps of: carrying out a reaction between the difluoroethanolation reagent B, cinnamic acid J, a catalyst D, and an oxidant E under stirring in an organic solvent F at 50-100° C. under nitrogen protection; after completion of the reaction, quenching the reaction by a tetrabutylammonium fluoride solution and then performing separating and purifying to obtain the α-difluoromethyl allyl alcohol compound K.

The reaction formula for the above method may be expressed as follows:

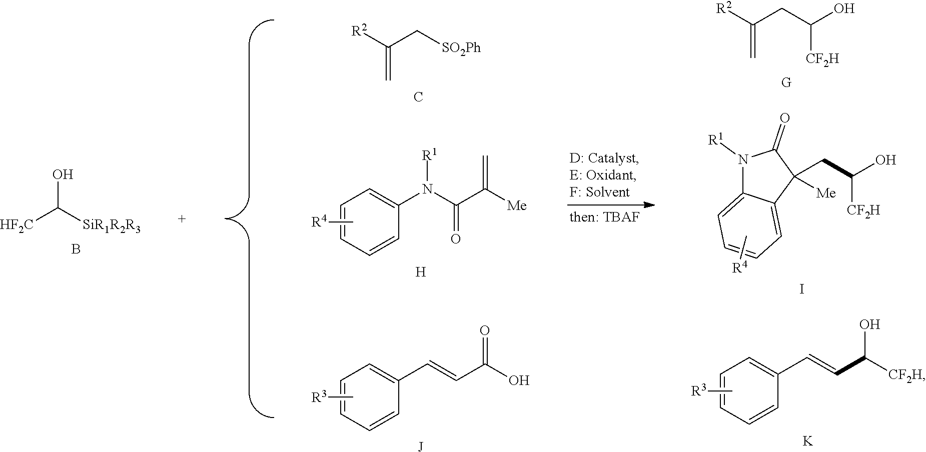

wherein, R², R³, and R⁴ groups represent substituent groups on allyl sulfone, cinnamic acid and acrylamide compounds, respectively, and include aryl, alkyl, aroyl, alkanoyl, substituted oxyacyl, alkoxy, halogen, alkoxy, etc., alkenyl, alkynyl, heteroaryl, and substituted sulfuryl.

The application of the fluorine-containing ethanolation reagents (A and B) of the present disclosure does not require additional bases, and may be subjected to a reaction with allyl sulfone, acrylamide, or cinnamic acid to obtain fluoromethyl homoallyl alcohol-, fluoromethyl alkyl alcohol-, and fluoromethyl allyl alcohol-containing compounds, respectively, by using manganese/peroxide as a catalytic system.

The present disclosure has the following beneficial effects: 1. the compound provided by the present disclosure is used as a trifluoroethanolation reagent (A) or a difluoroethanolation reagent (B) for synthesis intermediates of many organic compounds. Some of the compounds have pharmaceutical activity, and the preparation steps of such compounds are simplified, and the synthetic method has mild conditions and wide applicability of substrates; 2. the preparation of the trifluoroethanolation reagent (A) and the difluoroethanolation reagent (B) of the present disclosure may start from simple fluorine-containing ethanol to obtain the corresponding trifluoroacetyl silane and difluoroacetyl silane, followed by reduction with sodium borohydride, with mild reaction conditions and inexpensive and easily available raw materials. The construction of a trifluoroethanol α-C—Si bond is a key of the reagent of the present disclosure, and the ingenious use of radical brook rearrangement yields the cutting moiety of trifluoroethanol or difluoroethanol, which can be introduced into other specific molecules to achieve trifluoroethanolation and difluoroethanolation reactions.

### DETAILED DESCRIPTION OF THE EMBODIMENTS

The disclosure will be further described in detail below in conjunction with embodiments in order to enable a person skilled in the art to better understand the technical solutions of the present disclosure.

### Embodiment 1

Ethyl 5,5,5-trifluoro-4-hydroxy-2-methylene pentanoate, with a synthetic route and a preparation method as follows:

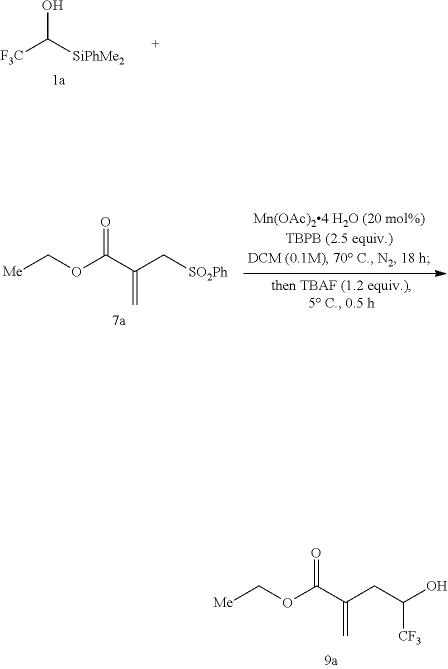

Under a nitrogen atmosphere, a magnetic stir bar was placed into a dry 10 mL Schlenk tube, and Mn(OAc)₂.4H₂O (14.7 mg, 0.06 mmol, 20 mol %), 7a (152.4 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) (PE: petroleum ether, EA: ethyl acetate) to give 53 mg (yield 71%) of the target compound as a yellow oil. The product was tested, with the results as follows:

R_{f}=0.23 (PE/EA=8/1, v/v).

NMR spectrum:

¹H NMR (400 MHz, CDCl₃) δ 6.35 (s, 1H), 5.80 (s, 1H), 4.25 (q, J=7.2 Hz, 2H), 4.16-4.08 (m, 1H), 3.63 (s, 1H), 2.78-2.57 (m, 2H), 1.32 (t, J=7.2 Hz, 3H);

¹³C NMR (100 MHz, CDCl₃) δ 168.1, 135.2, 129.8, 124.9 (q, J=280.5 Hz), 70.0 (q, J=30.7 Hz), 61.8, 33.5, 14.2;

¹⁹F NMR (375 MHz, CDCl₃) δ −79.6 (d, J=6.0 Hz, 3F).

IR (ATR): 3444, 2986, 2937, 1700, 1633, 1413, 1316, 1275, 1163, 1126, 1021, 712 cm⁻¹.

High resolution mass spectrum HRMS (ESI, m/z): a theoretically calculated value: C₈H₁₁F₃NaO₃⁺ (M+Na)⁺: 235.0552; found: 235.0556.

The trifluoroethanolation reagent 1a used in the embodiment was prepared as follows:

1) dimethylphenylsilyl chloride (30 mmol), trifluoroethanol (30 mmol), HMPA (6 mL) and tetrahydrofuran as a solvent were added into a dry single-neck flask with a magnetic stirrer, and the reaction flask was placed in a low-temperature tank of −78° C., and LDA (lithium diisopropylamide, 105 mmol) was added dropwise with a syringe pump. After completion of adding dropwise, stirring was kept to be performed for 4 h, the stirred material was heated to room temperature, and then stirring was performed again until trifluoroethanol was completely consumed. Triethylchlorosilane was added at 0° C., stirring was performed for 4 h, and the stirred material was separated by column chromatography to obtain a compound 4 (1,1-difluoro-2-dimethylphenylsilyl-2-triethylsiloxyethylene);

2) a fluorinating agent Select-Fluor (2.0 eq.) and a mixed solvent of acetonitrile and dichloromethane (4:1, v/v) were added to a dry single-neck flask with a magnetic stirrer, and the single-neck flask was placed in an ice-water bath of 0° C. The compound 4 was added to the reaction, and after completion, the reaction fluid was stirred at room temperature for 12 h, and the reaction was quenched with water, followed by separation by column chromatography to obtain a compound 5 (trifluoroacetyl phenyl dimethylsilane); and

(3) the compound 5 and methanol were added to the single-neck flask, and sodium borohydride solid was added to the reaction system in three batches, and after the reaction was completed, water was added to quench the reaction, followed by separation by column chromatography to obtain the trifluoroethanolation reagent 1a.

NMR spectrum of the trifluoroethanolation reagent 1a:

¹H NMR (400 MHz, CDCl3) δ 7.61 (d, J=6.1 Hz, 2H), 7.47-7.39 (m, 3H), 3.84 (q, J=11.1 Hz, 1H), 0.49 (d, J=3.1 Hz, 6H); ¹³C NMR (100 MHz, CDCl3) δ 134.4, 134.2, 130.3, 128.2, 127.1 (q, J=278.4 Hz), 65.3 (q, J=33.1 Hz), −4.7, −5.4; ¹⁹F NMR (375 MHz, CDCl3) δ −70.6 (d, J=8.9 Hz, 3F). IR (ATR): 3441, 2963, 2919, 1428, 1253, 1148, 1085, 1044, 738, 701 cm⁻¹.

HRMS (ESI, m/z): a theoretically calculated value: C₁₀H₁₃F₃NaO⁺ (M+Na)⁺: 257.0580; found: 257.0570.

### Embodiment 2

5,5,5-trifluoro-4-hydroxy-2-methylene-1-phenylpentan-1-one, with a synthetic route and a preparation method as follows:

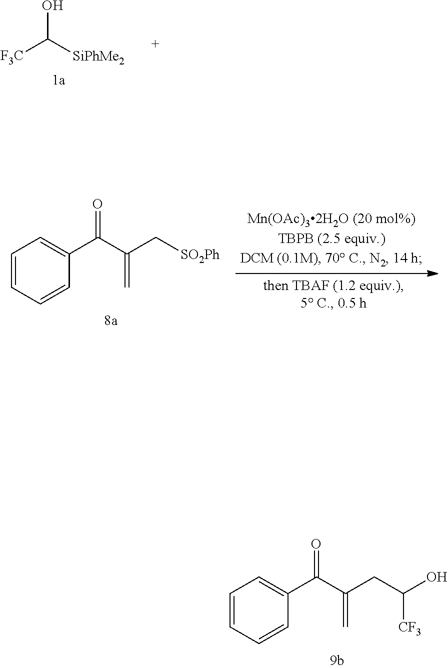

Under a nitrogen atmosphere, a magnetic stir bar was placed into a dry 10 mL Schlenk tube and Mn(OAc)₃.2H₂O (16.1 mg, 0.06 mmol, 20 mol %), 7b (257.4 mg, 0.9 mmol, 3.0 eq.), DCM (3 mL, 0.1 M), 1a (70.2 mg, 0.3 mmol)) and TBPB (145.7 mg, 0.75 mmol, 2.5 eq.) were added. Subsequently, the tube was sealed, and the mixture was heated to 70° C., and stirred for 14 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 53 mg (yield 71%) of the target compound as a yellow oil. The product was tested, with the results as follows:

R_{f}=0.40 (PE/EA=5/1, v/v).

NMR spectrum:

¹H NMR (400 MHz, CDCl₃) δ 7.77 (d, J=7.3 Hz, 2H), 7.60 (t, J=7.3 Hz, 1H), 7.47 (t, J=7.6 Hz, 2H), 6.16 (s, 1H), 5.90 (s, 1H), 4.32 (d, J=5.2 Hz, 1H), 4.15-4.14 (m, 1H), 2.90-2.71 (m, 2H);

¹³C NMR (100 MHz, CDCl₃) δ 199.5, 142.2, 136.7, 133.2, 131.9, 130.1, 128.5, 125.0 (q, J=280.6 Hz), 70.5 (q, J=31.2 Hz), 33.8; 19F NMR (375 MHz, CDCl₃) δ −79.3 (d, J=6.0 Hz, 3F).

IR (ATR): 3418, 3064, 2933, 1648, 1446, 1338, 1275, 1223, 1163, 1036, 753 cm⁻¹.

HRMS (ESI, m/z): a theoretically calculated value: C₁₂H₁₂F₃O₂⁺ (M+H)⁺: 245.0784; found: 245.0778.

### Embodiment 3

### 1-([[1,1′-biphenyl]-4-yl)-5,5,5-trifluoro-4-hydroxy-2-methylenepentyl-1-one

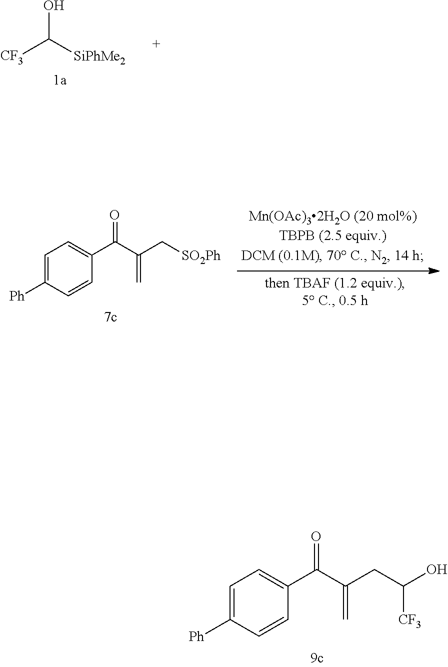

Under a nitrogen atmosphere, a magnetic stir bar was placed into a dry 10 mL Schlenk tube and Mn(OAc)₃.2H₂O (16.1 mg, 0.06 mmol, 20 mol %), 7c (326.2 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 74.9 mg (yield 78%) of the target compound as a white solid. The product was tested, with the results as follows:

R_{f}=0.50 (PE/EA=5/1, v/v), and a boiling point (mp): 69-71° C.

NMR spectrum:

¹H NMR (400 MHz, CDCl₃) δ 7.88 (d, J=8.3 Hz, 2H), 7.69 (d, J=8.3 Hz, 2H), 7.63 (d, J=7.3 Hz, 2H), 7.49 (t, J=7.3 Hz, 2H), 7.42 (t, J=7.2 Hz, 1H), 6.18 (s, 1H), 5.95 (s, 1H), 4.38 (d, J=4.9 Hz, 1H), 4.18-4.17 (m, 1H), 2.92-2.74 (m, 2H);

¹³C NMR (100 MHz, CDCl₃) δ 199.1, 146.1, 142.3, 139.8, 135.3, 131.5, 130.8, 129.1, 128.5, 127.4, 127.2, 125.0 (q, J=280.8 Hz), 70.6 (q, J=30.7 Hz), 33.9;

¹⁹F NMR (375 MHz, CDCl₃) δ −79.3 (d, J=6.0 Hz, 3F).

IR (ATR): 3392, 3060, 2926, 1640, 1599, 1409, 1344, 1275, 1163, 1129, 1029, 757 cm⁻¹.

HRMS (ESI, m/z): a theoretically calculated value: C₁₈H₁₆F₃O₂⁺ (M+H)⁺: 321.1097; found: 321.1096.

### Embodiment 4

### 1-(4-chlorophenyl)-5,5,5-trifluoro-4-hydroxy-2-methylenepentyl-1-one

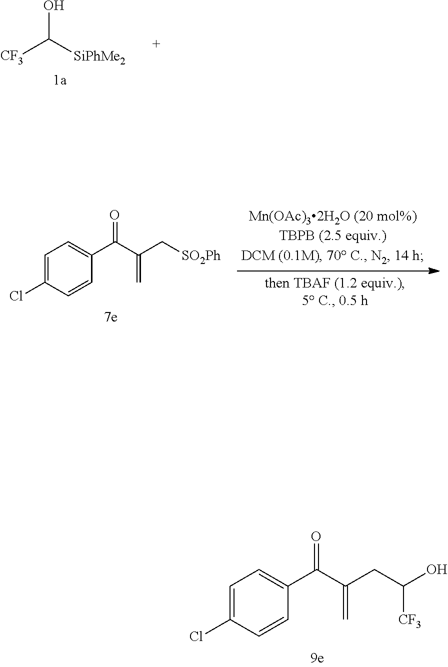

Under a nitrogen atmosphere, a magnetic stir bar was placed into a dry 10 mL Schlenk tube and Mn(OAc)₃.2H₂O (16.1 mg, 0.06 mmol, 20 mol %), 7e (288.0 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 57.2 mg (yield 69%) of the target compound as a yellow oil. The product was tested, with the results as follows:

R_{f}=0.40 (PE/EA=5/1, v/v).

NMR spectrum:

¹H NMR (400 MHz, CDCl₃) δ 7.73 (d, J=8.6 Hz, 2H), 7.45 (d, J=8.3 Hz, 2H), 6.16 (s, 1H), 5.87 (s, 1H), 4.15 (s, 1H), 3.96 (s, 1H), 2.90 2.71 (m, 2H);

¹³C NMR (100 MHz, CDCl₃) δ 198.1, 142.1, 139.7, 135.0, 131.7, 131.4, 128.9, 124.9 (q, J=281.8 Hz), 70.4 (q, J=30.7 Hz), 33.8; ¹⁹F NMR (375 MHz, CDCl₃) δ −79.4 (d, J=8.9 Hz, 3F).

IR (ATR): 3437, 2930, 1651, 1588, 1478, 1402, 1334, 1275, 1163, 1129, 1092, 790 cm⁻¹.

HRMS (ESI, m/z): a theoretically calculated value: C₁₂H₁₁ClF₃O₂⁺ (M+H)⁺: 279.0394; found: 279.0389.

### Embodiment 5

### 1-(3-chlorophenyl)-5,5,5-trifluoro-4-hydroxy-2-methylenepentyl-1-one

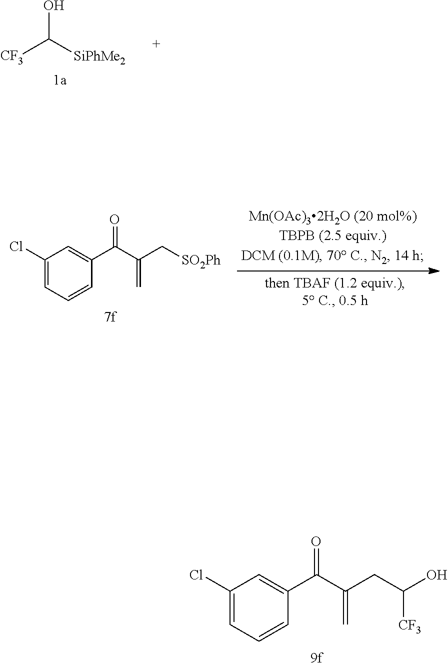

Under a nitrogen atmosphere, a magnetic stir bar was placed into a dry 10 mL Schlenk tube and Mn(OAc)₃.2H₂O (16.1 mg, 0.06 mmol, 20 mol %), 7f (288.0 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 65.1 mg (yield 78%) of the target compound as a yellow oil. The product was tested, with the results as follows:

R_{f}=0.40 (PE/EA=5/1, v/v).

NMR spectrum:

¹H NMR (400 MHz, CDCl₃) δ 7.73 (s, 1H), 7.63 (d, J=8.0 Hz, 1H), 7.56 (d, J=8.0 Hz, 1H), 7.41 (t, J=7.8 Hz, 1H), 6.19 (s, 1H), 5.90 (s, 1H), 4.19 4.13 (m, 1H), 3.85 (d, J=5.8 Hz, 1H), 2.91-2.71 (m, 2H);

¹³C NMR (100 MHz, CDCl₃) δ 197.9, 142.0, 138.5, 134.8, 133.0, 132.3, 129.9, 129.9, 128.0, 124.9 (q, J=280.8 Hz), 70.2 (q, J=30.9 Hz), 33.6;

¹⁹F NMR (375 MHz, CDCl₃) δ −79.4 (d, J=6.0 Hz, 3F). IR (ATR): 3418, 3071, 2930, 1651, 1420, 1334, 1275, 1163, 1129, 1033, 768 cm⁻¹.

HRMS (ESI, m/z): a theoretically calculated value: C₁₂H₁₀ClF₃NaO₂⁺ (M+Na)⁺: 301.0214; found: 301.0216.

### Embodiment 6

### 1-(4-bromophenyl)-5,5,5-trifluoro-4-hydroxy-2-methylenepentyl-1-one

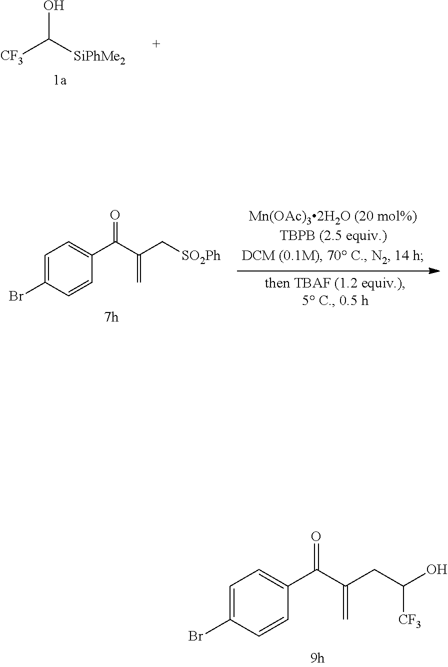

Under a nitrogen atmosphere, a magnetic stir bar was placed into a dry 10 mL Schlenk tube and Mn(OAc)₃.2H₂O (16.1 mg, 0.06 mmol, 20 mol %), 7h (329.0 mg, 0.9 mmol, 3.0 eq.), DCM (3 mL 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, TBAF was added to the stirred material in an ice-water bath of 5° C. to be stirred for 0.5 h. The reaction mixture was quenched with water (2 mL), and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 78.9 mg (yield 81%) of the target compound as a yellow oil. The product was tested, with the results as follows:

R_{f}=0.50 (PE/EA=5/1, v/v).

NMR spectrum:

¹H NMR (400 MHz, CDCl₃) δ 7.66-7.60 (m, 4H), 6.17 (s, 1H), 5.87 (s, 1H), 4.15 4.14 (m, 1H), 3.92-3.91 (m, 1H), 2.90-2.71 (m, 2H);

¹³C NMR (100 MHz, CDCl₃) δ 198.3, 142.1, 135.5, 131.9, 131.8, 131.5, 128.4, 124.9 (q, J=280.8 Hz), 70.4 (q, J=30.9 Hz), 33.7;

¹⁹F NMR (375 MHz, CDCl₃) δ −79.4 (d, J=6.0 Hz, 3F).

IR (ATR): 3422, 2920, 2855, 1648, 1584, 1398, 1275, 1167, 1133, 1074, 790 cm⁻¹.

HRMS (ESI, m/z): a theoretically calculated value: C₁₂H₁₁BrF₃O₂⁺ (M+H)⁺: 322.9889; found: 322.9888.

### Embodiment 7

### 5,5,5-trifluoro-4-hydroxy-1-(4-iodophenyl)-2-methylenepentyl-1-one

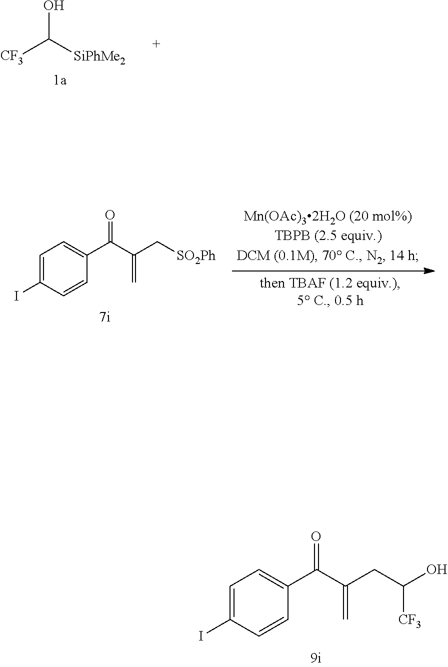

Under a nitrogen atmosphere, a magnetic stir bar was placed into a dry 10 mL Schlenk tube and Mn(OAc)₃.2H₂O (16.1 mg, 0.06 mmol, 20 mol %), 7i (370.8 mg, 0.9 mmol, 3.0 eq.), DCM (3 mL, 0.1 M), 1a (70.2 mg, 0.3 mmol)) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was then quenched with water (2 mL) and subjected to extraction with DCM (3×10 mL), and the organic phases were mixed and washed with brine, dried over Na₂SO₄ and evaporated to dryness by using a rotary evaporator. The crude product was purified by silica gel column chromatography (200×300-mesh) and eluted with PE/EA (20/1-10/1, v/v) to give 72.0 mg (yield 65%) of the target compound as a colorless oil. The product was tested, with the results as follows:

R_{f}=0.50 (PE/EA=5/1, v/v).

NMR spectrum:

¹H NMR (400 MHz, CDCl₃) δ 7.83 (d, J=8.3 Hz, 2H), 7.48 (d, J=8.3 Hz, 2H), 6.16 (s, 1H), 5.86 (s, 1H), 4.14 (s, 1H), 3.95 (d, J=4.6 Hz, 1H), 2.89 2.70 (m, 2H);

¹³C NMR (100 MHz, CDCl₃) δ 198.5, 142.1, 137.9, 136.0, 131.8, 131.4, 124.9 (q, J=280.8 Hz), 101.0, 70.3 (q, J=31.1 Hz), 33.8;

¹⁹F NMR (375 MHz, CDCl₃) δ −79.4 (d, J=6.0 Hz, 3F).

IR (ATR): 3429, 2926, 2855, 1648, 1480, 1390, 1275, 1163, 1126, 1100, 787 cm⁻¹.

HRMS (ESI, m/z): a theoretically calculated value: C₁₂H₁₀F₃INaO₂⁺ (M+Na)⁺: 392.9570; found: 392.9560.

### Embodiment 8

### 5,5,5-trifluoro-4-hydroxy-2-methylene pentanoic acid-4-bromo-2-ene-butyl Ester

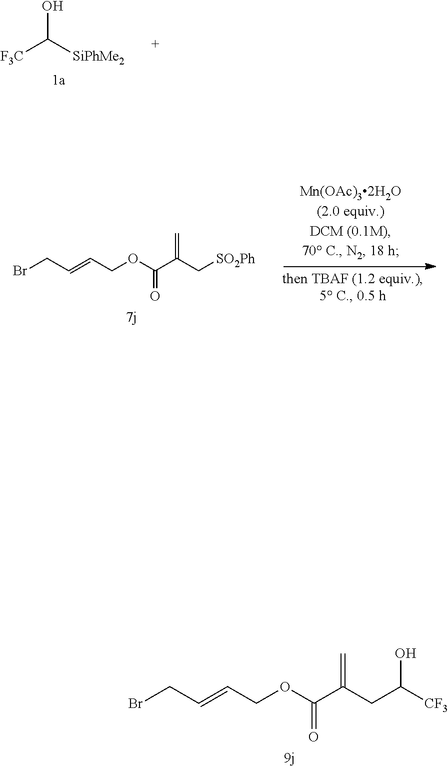

Under a nitrogen atmosphere, a magnetic stir bar was placed into a dry 10 mL Schlenk tube and Mn(OAc)₃.2H₂O (16.1 mg, 0.06 mmol, 20 mol %), 7j (323.1 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 45.7 mg (yield 48%) of the target compound as a colorless oil. The product was tested, with the results as follows:

R_{f}=0.57 (PE/EA=4/1 v/v).

NMR spectrum:

¹H NMR (400 MHz, CDCl₃) δ 6.39 (s, 1H), 5.95-5.92 (m, 2H), 5.85 (s, 1H), 4.71 (d, J=4.3 Hz, 2H), 4.18-4.07 (m, 3H), 3.28 (s, 1H), 2.80-2.58 (m, 2H);

¹³C NMR (100 MHz, CDCl₃) δ 167.5, 134.9, 130.4, 130.3, 127.9, 124.9 (q, J=281.2 Hz), 69.9 (q, J=31.2 Hz), 64.7, 43.9, 33.5.

¹⁹F NMR (375 MHz, CDCl₃) δ −79.6 (d, J=8.9 Hz, 3F).

IR (ATR): 3429, 2922, 2855, 2359, 2259, 1715, 1126, 783 cm⁻¹.

HRMS (ESI, m/z): a theoretically calculated value: C₁₀H₁₂F₃O₃⁺ (M-Br)⁺: 237.0733; found: 237.0726.

### Embodiment 9

### 5,5,5-trifluoro-1-(furan-2-yl)-4-hydroxy-2-methylenepentyl-1-one

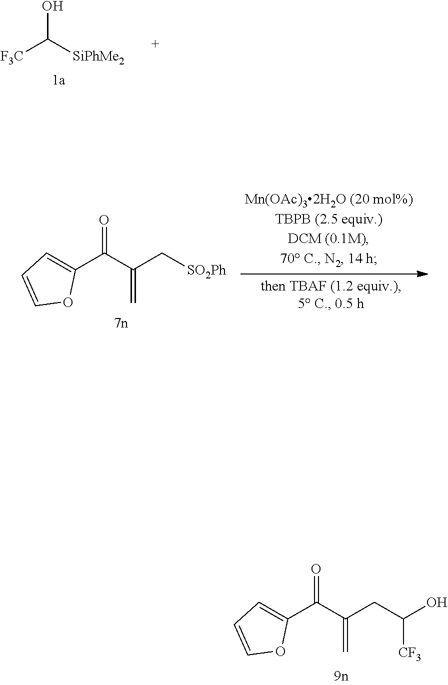

Under a nitrogen atmosphere, a magnetic stir bar was placed into a dry 10 mL Schlenk tube and Mn(OAc)₃.2H₂O (16.1 mg, 0.06 mmol, 20 mol %), 7n (248.7 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 56.0 mg (yield 80%) of the target compound as a yellow oil. The product was tested, with the results as follows:

R_{f}=0.40 (PE/EA=5/1, v/v).

NMR spectrum:

¹H NMR (400 MHz, CDCl₃) δ 7.71-7.70 (m, 1H), 7.26-7.25 (m, 1H), 6.59-6.58 (m, 1H), 6.27 (s, 1H), 6.07 (s, 1H), 4.15-4.07 (m, 1H), 2.83-2.65 (m, 2H);

¹³C NMR (100 MHz, CDCl₃) δ 184.7, 151.4, 148.3, 141.8, 130.0, 124.9 (q, J=280.8 Hz), 121.9, 112.6, 70.5 (q, J=31.2 Hz), 34.0;

¹⁹F NMR (375 MHz, CDCl₃) δ −79.4 (d, J=6.0 Hz, 3F).

IR (ATR): 3407, 3142, 2933, 1618, 1465, 1394, 1275, 1163, 1129, 1029, 768 cm⁻¹.

HRMS (ESI, m/z): a theoretically calculated value: C₁₀H₁₀F₃O₃⁺ (M+H)⁺: 235.0577; found: 235.0575.

### Embodiment 10

### N,N-diphenyl 5,5,5-trifluoro-4-hydroxy-2-methylene Pentanamide

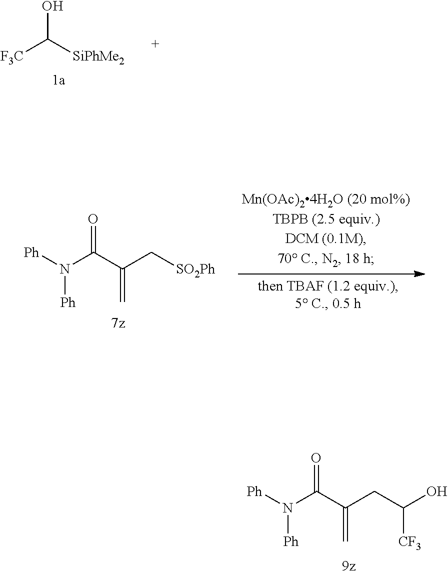

Under a nitrogen atmosphere, a magnetic stir bar was placed into a dry 10 mL Schlenk tube and Mn(OAc)₂.4H₂O (14.7 mg, 0.06 mmol, 20 mol %), 7z (339.3 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (8/1, v/v) to give 54 mg (yield 54%) of the target compound as a colorless oil. The product was tested, with the results as follows:

R_{f}=0.27 (PE/EA=4/1, v/v).

NMR spectrum:

¹H NMR (400 MHz, CDCl₃) δ 7.37 (t, J=7.8 Hz, 4H), 7.29-7.25 (m, 2H), 7.19-7.17 (m, 4H), 5.45 (s, 1H), 5.32 (s, 1H), 4.15-4.07 (m, 1H), 2.64-2.50 (m, 2H);

¹³C NMR (100 MHz, CDCl₃) δ 172.4, 143.2, 138.6, 129.5, 127.3, 127.2, 125.8, 125.0 (q, J=280.3 Hz), 71.1 (q, J=30.7 Hz), 34.8; ¹⁹F NMR (375 MHz, CDCl₃) δ −79.5 (d, J=6.0 Hz, 3F).

IR (ATR): 3288, 2963, 2930, 1644, 1592, 1491, 1364, 1275, 1163, 1126, 1029, 693 cm⁻¹.

HRMS (ESI, m/z): a theoretically calculated value: C₁₈H₁₇F₃NO₂⁺ (M+H)⁺: 336.1206; found: 336.1197.

### Embodiment 11

### 4-(5,5,5-trifluoro-4-hydroxy-2-methylene valeryl)benzonitrile

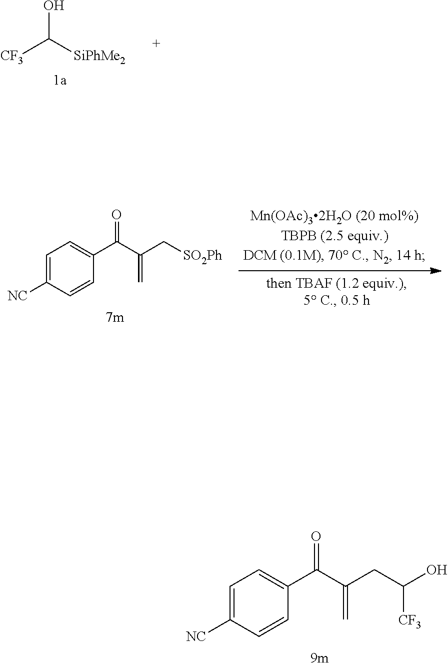

Under a nitrogen atmosphere, a magnetic stir bar was placed into a dry 10 mL Schlenk tube and Mn(OAc)₃.2H₂O (16.1 mg, 0.06 mmol, 20 mol %), 7m (280.2 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 65.0 mg (yield 80%) of the target compound as a yellow oil. The product was tested, with the results as follows:

R_{f}=0.30 (PE/EA=5/1, v/v).

NMR spectrum:

¹H NMR (400 MHz, CDCl₃) δ 7.83 (d, J=8.6 Hz, 2H), 7.77 (d, J=8.6 Hz, 2H), 6.24 (s, 1H), 5.86 (s, 1H), 4.23-4.15 (m, 1H), 3.42 (s, 1H), 2.95-2.74 (m, 2H);

¹³C NMR (100 MHz, CDCl₃) δ 197.4, 142.0, 140.7, 132.9, 132.4, 130.2, 124.9 (q, J=280.3 Hz), 118.0, 116.1, 69.9 (q, J=30.9 Hz), 33.3;

¹⁹F NMR (375 MHz, CDCl₃) δ −79.4 (d, J=6.0 Hz, 3F).

IR (ATR): 3448, 2922, 2851, 2233, 1655, 1402, 1275, 1163, 1129, 1029, 798 cm⁻¹.

HRMS (ESI, m/z): a theoretically calculated value: C₁₃H₁₀F₃NNaO₂⁺ (M+Na)⁺: 292.0556; found: 292.0567.

### Embodiment 12

### 4-ene-4-phenyl-1,1,1-trifluoropentan-2-ol

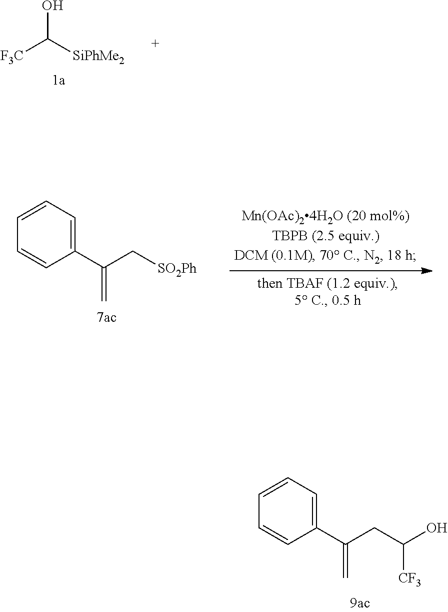

Under a nitrogen atmosphere, a magnetic stir bar was placed into a dry 10 mL Schlenk tube and Mn(OAc)₂.4H₂O (29.4 mg, 0.12 mmol, 20 mol %), 7ac (465.0 mg, 1.8 mmol, 3.0 eq.), DCM (6 mL, 0.1 M), 1a (140.4 mg, 0.6 mmol) and TBPB (291.3 mg, 1.5 mmol, 2.5 eq) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (30/1-20/1, v/v) to give 56 mg (yield 43%) of the target compound as a yellow oil. The product was tested, with the results as follows:

R_{f}=0.47 (PE/EA=8/1, v/v).

NMR spectrum:

¹H-NMR (400 MHz, CDCl₃) δ 7.42-7.31 (m, 5H), 5.49 (s, 1H), 5.28 (s, 1H), 4.00 (bs, 1H), 3.11-2.67 (m, 2H), 2.21 (s, 1H);

¹³C-NMR (100 MHz, CDCl₃) δ 142.7, 139.4, 128.8, 128.3, 126.3, 125.2 (q, J=279.8 Hz), 121.1, 117.0, 68.7 (q, J=30.9 Hz), 36.3;

¹⁹F-NMR (375 MHz, CDCl₃) δ −79.5 (d, J=6.0 Hz, 3F).

IR (ATR): 3422, 3086, 3030, 2960, 2930, 1633, 1446, 1390, 1029, 701 cm⁻¹.

HRMS (ESI, m/z): a theoretically calculated value: C₁₁H₁₂F₃O⁺ (M+H)⁺: 217.0835; found: 217.0828.

### Embodiment 13

Epiandrosterone Derivative

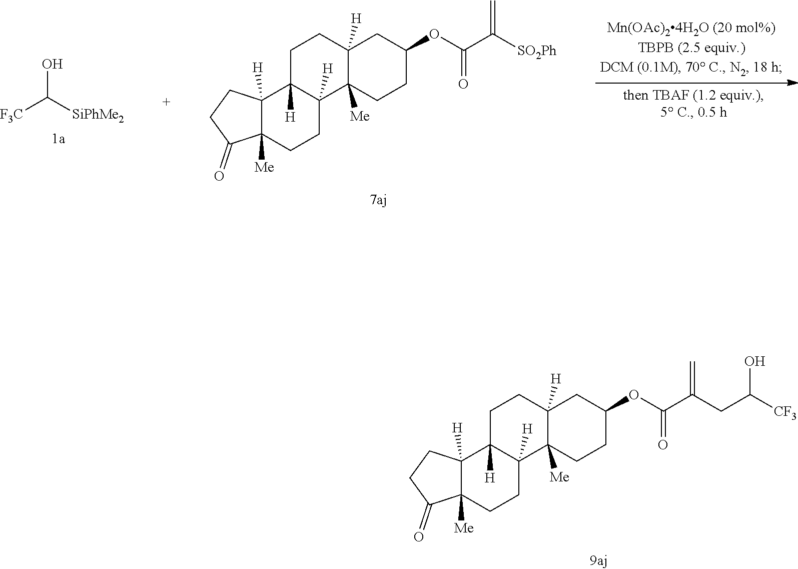

Under a nitrogen atmosphere, a magnetic stir bar was placed into a dry 10 mL Schlenk tube and added Mn(OAc)₂.4H₂O (14.7 mg, 0.06 mmol, 20 mol %), 7aj (298.8 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (5/1, v/v) to give 81 mg (yield 59%) of the target compound as a white solid. The product was tested, with the results as follows:

R_{f}=0.24 (PE/EA=2/1, v/v). mp: 95-97° C.

NMR spectrum:

¹H NMR (400 MHz, CDCl₃) δ 6.31 (s, 1H), 5.77 (s, 1H), 4.81-4.73 (m, 1H), 4.11-4.08 (m, 1H), 3.82 (s, 1H), 2.76-2.55 (m, 2H), 2.46-2.39 (m, 1H), 2.11-2.01 (m, 1H), 1.95-1.75 (m, 5H), 1.67-1.19 (m, 12H), 1.09-0.96 (m, 2H), 0.86 (s, 3H), 0.85 (s, 3H), 0.75-0.69 (m, 1H);

¹³C NMR (100 MHz, CDCl₃) δ 221.7, 167.5, 135.5, 129.5, 124.9 (q, J=280.8 Hz), 75.1, 70.0 (q, J=30.9 Hz), 54.3, 51.4, 47.9, 44.7, 36.7, 36.0, 35.7, 35.1, 33.9, 33.5, 31.6, 30.9, 28.3, 27.4, 21.9, 20.6, 13.9, 12.3;

¹⁹F NMR (375 MHz, CDCl₃) δ −79.5-−79.5 (m, 3F, 3F′).

IR (ATR): 3370, 2933, 2855, 1718, 1633, 1405, 1312, 1291, 1178, 1122, 1014, 716 cm⁻¹.

HRMS (ESI, m/z): a theoretically calculated value: C₂₅H₃₅F₃NaO₄⁺ (M+Na)⁺: 479.2380; found: 479.2370.

### Embodiment 14

### 5,5,5-trifluoro-4-hydroxy-2-methylene Pentanoic Acid-indan-1-yl Ester

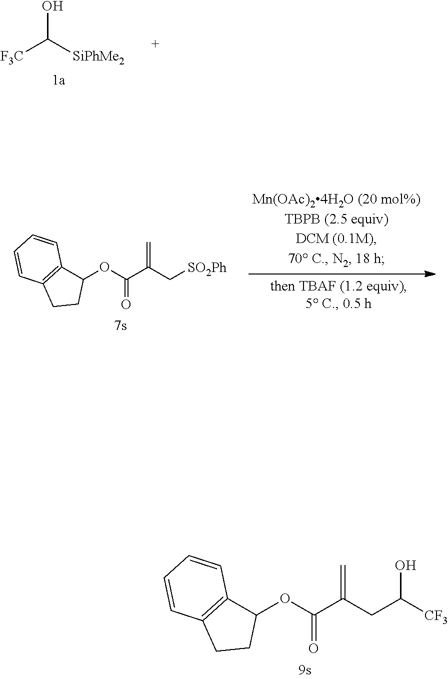

Under a nitrogen atmosphere, a magnetic stir bar was placed into a dry 10 mL Schlenk tube and Mn(OAc)₂.4H₂O (14.7 mg, 0.06 mmol, 20 mol %), 7a (152.4 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 66 mg (yield 73%) of the target compound as a colorless oil. The product was tested, with the results as follows:

R_{f}=0.30 (PE/EA=8/1, v/v).

NMR spectrum:

¹H NMR (400 MHz, CDCl₃) δ 7.43 (d, J=7.3 Hz, 1H), 7.35-7.31 (m, 2H), 7.25 (t, J=6.3 Hz, 1H), 6.32-6.28 (m, 2H), 5.79 (s, 1H), 4.14-4.12 (m, 1H), 3.67 (s, 1H), 3.18 3.11 (m, 1H), 2.96-2.89 (m, 1H), 2.80-2.76 (m, 1H), 2.66-2.52 (m, 2H), 2.20-2.13 (m, 1H);

¹³C NMR (100 MHz, CDCl₃) δ 168.1, 144.6, 140.6, 135.3, 130.1, 130.0 (C′), 129.4, 127.0, 125.8, 125.7 (C′), 125.0, 124.9 (q, J=280.8 Hz), 79.9, 71.2 (q, J=30.9 Hz, C), 70.1 (q, J=31.2 Hz, C′), 33.6, 32.4, 30.3;

¹⁹F NMR (375 MHz, CDCl₃) δ −79.5-−79.5 (m, 3F, 3F′).

IR (ATR): 3425, 2941, 2855, 1703, 1633, 1435, 1320, 1275, 1170, 1126, 1014, 708 cm⁻¹.

HRMS (ESI, m/z): a theoretically calculated value: C₁₅H₁₅F₃NaO₃⁺ (M+Na)⁺: 323.0866; found: 323.0867.

### Embodiment 15

### 5,5,5-trifluoro-4-hydroxy-2-methylenepentanoic Acid benzo[d][1,3]dioxolan-5-yl Methyl Ester

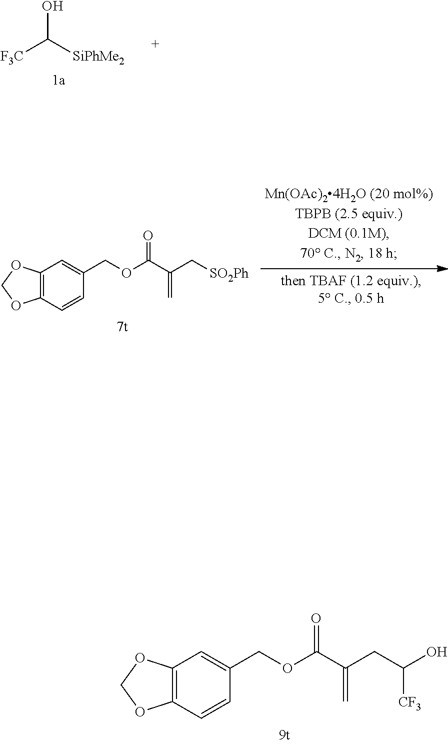

Under a nitrogen atmosphere, a magnetic stir bar was placed into a dry 10 mL Schlenk tube and Mn(OAc)₂.4H₂O (14.7 mg, 0.06 mmol, 20 mol %), 7t (216 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL), and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (8/1, v/v) to give 61 mg (yield 64%) of the target compound as a colorless oil. The product was tested, with the results as follows:

R_{f}=0.33 (PE/EA=4/1, v/v).

NMR spectrum:

¹H NMR (400 MHz, CDCl₃) δ 6.85-6.77 (m, 3H), 6.35 (s, 1H), 5.96 (s, 2H), 5.81 (s, 1H), 5.11 (s, 2H), 4.15-4.07 (m, 1H), 3.10 (s, 1H), 2.78-2.56 (m, 2H);

¹³C NMR (100 MHz, CDCl₃) δ 167.7, 148.0, 147.8, 135.0, 130.2, 129.2, 124.9 (q, J=280.0 Hz), 122.5, 109.1, 108.4, 101.4, 69.9 (q, J=31.1 Hz), 67.4, 33.5;

¹⁹F NMR (375 MHz, CDCl₃) δ −79.6 (d, J=6.0 Hz, 3F).

IR (ATR): 3422, 2900, 1707, 1633, 1491, 1446, 1327, 1252, 1167, 1122, 1036, 712 cm⁻¹.

HRMS (ESI, m/z): a theoretically calculated value: C₁₄H₁₃F₃NaO₅⁺ (M+Na)⁺: 341.0607; found: 341.0594.

### Embodiment 16

### 5,5,5-trifluoro-4-hydroxy-2-methylenepentanoic acid-4-bromobenzyl Ester

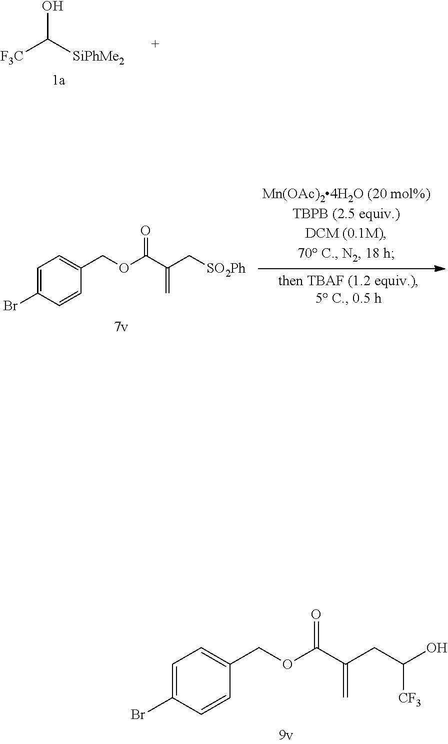

Under a nitrogen atmosphere, a magnetic stir bar was placed into a dry 10 mL Schlenk tube and Mn(OAc)₂.4H₂O (14.7 mg, 0.06 mmol, 20 mol %), 7v (355.5 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄ and evaporated to dryness by using a rotary evaporator. The crude product was purified by silica gel column chromatography (200×300-mesh) and eluted with PE/EA (20/1-10/1, v/v) to give 60 mg (yield 56%) of the target compound as a yellow oil. The product was tested, with the results as follows:

R_{f}=0.23 (PE/EA=4/1, v/v).

NMR spectrum:

¹H NMR (400 MHz, CDCl₃) δ 7.51 (d, J=8.3 Hz, 2H), 7.25 (d, J=8.4 Hz, 2H), 6.39 (s, 1H), 5.85 (s, 1H), 5.17 (s, 2H), 4.17-4.09 (m, 1H), 2.81-2.59 (m, 3H);

¹³C NMR (100 MHz, CDCl₃) δ 167.6, 134.8, 134.5, 132.0, 130.4, 130.1, 124.9 (q, J=279.8 Hz), 122.7, 69.9 (q, J=31.1 Hz), 66.6, 33.4;

¹⁹F NMR (375 MHz, CDCl₃) δ −79.6 (d, J=6.0 Hz, 3F).

IR (ATR): 3422, 3528, 2498, 1715, 1633, 1439, 1331, 1275, 1170, 1126, 1014, 712 cm⁻¹.

HRMS (ESI, m/z): a theoretically calculated value: C₁₃H₁₂BrF₃NaO₃⁺ (M+Na)⁺: 374.9814; found: 374.9807.

### Embodiment 17

### 5,5,5-trifluoro-4-hydroxy-2-methylenepentanoic acid-naphthalene-2-methyl Ester

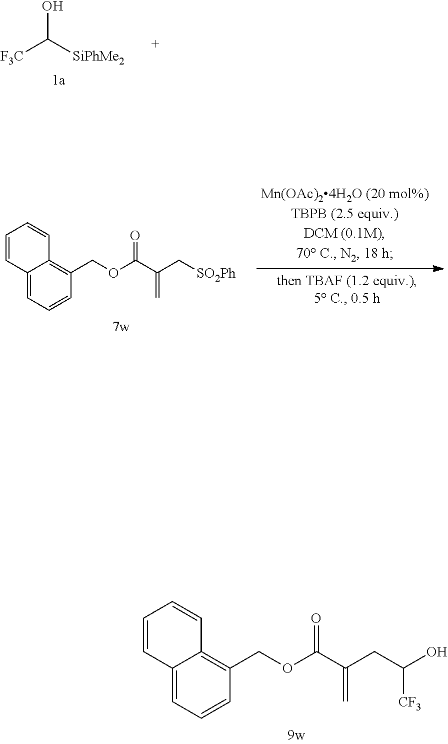

Under a nitrogen atmosphere, a magnetic stir bar was placed into a dry 10 mL Schlenk tube and Mn(OAc)₂.4H₂O (14.7 mg, 0.06 mmol, 20 mol %), 7w (329.8 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, TBAF was added to the stirred material in an ice-water bath of 5° C. to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 70 mg (yield 72%) of the target compound as a colorless oil. The product was tested, with the results as follows:

R_{f}=0.3 (PE/EA=10/1, v/v).

NMR spectrum:

¹H NMR (400 MHz, CDCl₃) δ 8.03 (d, J=7.9 Hz, 1H), 7.89 (t, J=8.9 Hz, 2H), 7.60-7.52 (m, 3H), 7.47 (t, J=7.5 Hz, 1H), 6.35 (s, 1H), 5.80 (s, 1H), 5.70 (s, 2H), 4.17-4.09 (m, 1H), 3.31 (s, 1H), 2.81-2.59 (m, 2H);

¹³C NMR (100 MHz, CDCl₃) δ 167.8, 134.9, 133.9, 131.8, 131.0, 130.5, 129.7, 128.95, 127.87, 126.86, 126.19, 125.40, 124.9 (q, J=280.8 Hz), 123.5, 69.9 (q, J=30.7 Hz), 65.8, 33.5;

¹⁹F NMR (375 MHz, CDCl₃) δ −79.6 (d, J=6.0 Hz, 3F).

IR (ATR): 3444, 3049, 2937, 1707, 1633, 1413, 1320, 1271, 1167, 1126, 1029, 775 cm⁻¹.

HRMS (ESI, m/z): a theoretically calculated value: C₁₇H₁₅F₃NaO₃⁺ (M+Na)⁺: 347.0866; found: 347.0875.

### Embodiment 18

### N,N-diphenyl 5,5,5-trifluoro-4-hydroxy-2-methylenepentanamide

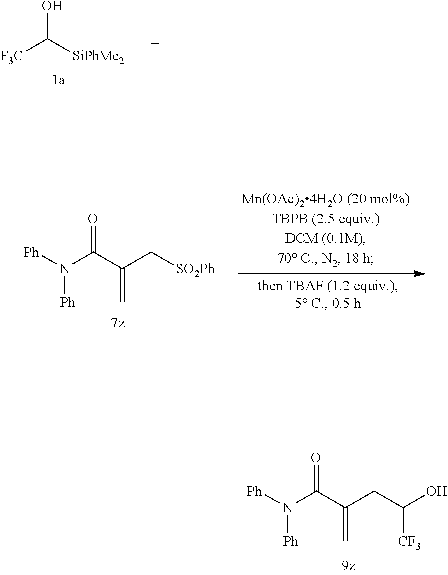

Under a nitrogen atmosphere, a magnetic stir bar was placed into a dry 10 mL Schlenk tube and Mn(OAc)₂.4H₂O (14.7 mg, 0.06 mmol, 20 mol %), 7z (339.3 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (8/1, v/v) to give 54 mg (yield 54%) of the target compound as a colorless oil. The product was tested, with the results as follows:

R_{f}=0.27 (PE/EA=4/1, v/v).

NMR spectrum:

¹H NMR (400 MHz, CDCl₃) δ 7.37 (t, J=7.8 Hz, 4H), 7.29-7.25 (m, 2H), 7.19-7.17 (m, 4H), 5.45 (s, 1H), 5.32 (s, 1H), 4.15-4.07 (m, 1H), 2.64-2.50 (m, 2H);

¹³C NMR (100 MHz, CDCl₃) δ 172.4, 143.2, 138.6, 129.5, 127.3, 127.2, 125.8, 125.0 (q, J=280.3 Hz), 71.1 (q, J=30.7 Hz), 34.8;

¹⁹F NMR (375 MHz, CDCl₃) δ −79.5 (d, J=6.0 Hz, 3F).

IR (ATR): 3288, 2963, 2930, 1644, 1592, 1491, 1364, 1275, 1163, 1126, 1029, 693 cm⁻¹.

HRMS (ESI, m/z): a theoretically calculated value: C₁₈H₁₇F₃NO₂⁺ (M+H)⁺: 336.1206; found: 336.1197.

### Embodiment 19

### N,N-dimethyl 5,5,5-trifluoro-4-hydroxy-2-methylenepentanamide

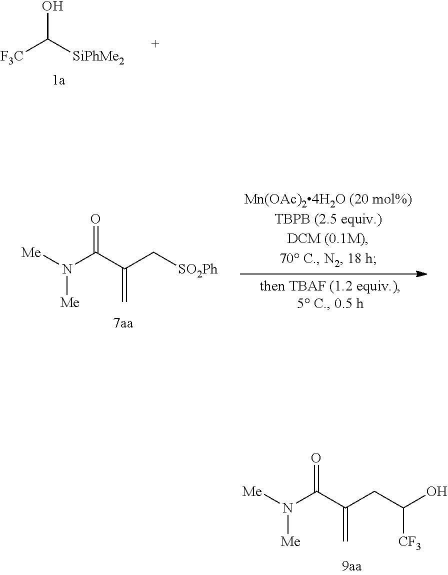

Under a nitrogen atmosphere, a magnetic stir bar was placed into a dry 10 mL Schlenk tube and Mn(OAc)₃.2H₂O (16.1 mg, 0.06 mmol, 20 mol %), 7aa (227.9 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL), and the organic phases were mixed and washed with brine, dried over Na₂SO₄, and evaporated to dryness by using a rotary evaporator. The crude product was purified by silica gel column chromatography (200×300-mesh) and eluted with PE/EA (10/1, v/v) to give 40 mg (63%) of the target compound as a yellow oil. The product was tested, with the results as follows:

R_{f}=0.30 (PE/EA=2/1, v/v).

NMR spectrum:

¹H NMR (400 MHz, CDCl₃) δ 5.58 (s, 1H), 5.37 (s, 1H), 4.10-4.02 (m, 1H), 3.14 (s, 3H), 3.03 (s, 3H), 2.64-2.41 (m, 2H);

¹³C NMR (100 MHz, CDCl₃) δ 172.4, 137.6, 125.1 (q, J=280.3 Hz), 121.8, 71.1 (q, J=30.7 Hz), 39.8, 35.5, 34.8;

¹⁹F NMR (375 MHz, CDCl₃) δ −79.4 (d, J=6.0 Hz, 3F).

IR (ATR): 3329, 2930, 1610, 1454, 1264, 1167, 1118, 1029, 734 cm⁻¹

HRMS (ESI, m/z): a theoretically calculated value: C₈H₁₂F₃NNaO₂⁺ (M+Na)⁺: 234.0712; found: 234.0707.

### Embodiment 20

### 4-ene-4-(4-methylbenzenesulfonyl)-1,1,1-trifluoro-pentan-2-ol

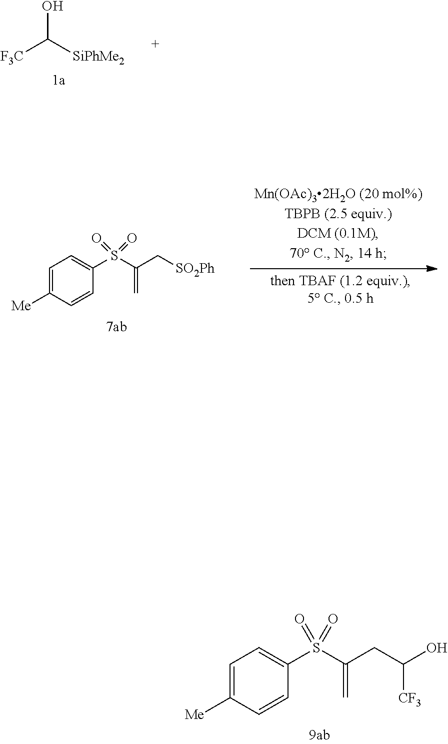

Under a nitrogen atmosphere, a magnetic stir bar was placed into a dry 10 mL Schlenk tube and Mn(OAc)₃.2H₂O (16.1 mg, 0.06 mmol, 20 mol %), 7ab (302.7 mg, 0.9 mmol, 3.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 14 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 63.5 mg (yield 72%) of the target compound as a yellow oil. The product was tested, with the results as follows:

R_{f}=0.30 (PE/EA=5/1, v/v).

NMR spectrum:

¹H NMR (400 MHz, CDCl₃) δ 7.76 (d, J=8.4 Hz, 2H), 7.37 (d, J=8.0 Hz, 2H), 6.47 (s, 1H), 5.96 (s, 1H), 4.30-4.22 (m, 1H), 2.63-2.46 (m, 5H);

¹³C NMR (100 MHz, CDCl₃) δ 145.5, 145.0, 134.7, 130.3, 128.6, 128.3, 124.6 (q, J=277.9 Hz), 69.1 (q, J=31.4 Hz), 31.2, 21.8;

¹⁹F NMR (375 MHz, CDCl₃) δ −79.7 (d, J=6.0 Hz, 3F).

IR (ATR): 3474, 2930, 2855, 1595, 1431, 1279, 1137, 1081, 734 cm⁻¹.

HRMS (ESI, m/z): a theoretically calculated value: C₁₂H₁₃F₃NaO₃S⁺ (M+Na)⁺: 317.0430; found: 317.0432.

### Embodiment 21

### 3-methyl-1-phenyl-3-(3,3,3-trifluoro-2-hydroxypropyl)indol-2-one

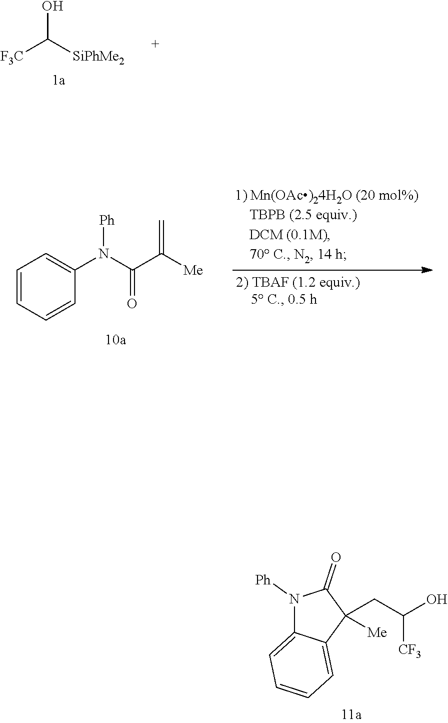

Under a nitrogen protection, Mn(OAc)₂.4H₂O (29.4 mg, 0.12 mmol, 20 mol %) and 10a (170.7 mg, 0.72 mmol, 1.2 eq.) were added into a dry 25 mL Schlenk reaction tube containing a polytetrafluoroethylene magnetic stir bar with a suitable size, followed by addition of DCM (6 mL, 0.1 M), 1a (140.4 mg, 0.6 mmol) and TBPB (291.5 mg, 1.5 mmol, 2.5 eq.). The reaction tube was sealed, and then placed on a heating module, and the mixture was heated to 70° C. to be subjected to a reaction for 14 h. The reaction tube was taken down. After cooled to room temperature, the reaction tube was put into an ice water bath, and opened, TBAF (188.3 mg, 0.72 mmol, 1.2 eq.) was added, and then the reaction tube was sealed. The reaction was carried out under stirring in the ice water bath for 30 min, and quenched with 8 mL of water, and the reaction product was subjected to extraction with saturated NaCl (30 mL) and DCM (3×20 mL). The organic phases were mixed, then washed with saturated sodium chloride (2×50 mL), dried over anhydrous Na₂SO₄, filtered, and concentrated with a rotary evaporator under reduced pressure to obtain a crude product. The resulting crude product was separated by silica gel column chromatography with petroleum ether and ethyl acetate as an eluent to obtain 3-methyl-1-phenyl-3-(3,3,3-trifluoro-2-hydroxypropyl)indol-2-one (a total yield of two diastereoisomers is 910%). The product was tested, with the results as follows:

The diastereoisomer with larger polarity: R_{f}=0.32 (PE/EA=5/1 v/v). mp: 116-118° C.

NMR spectrum:

¹H NMR (400 MHz, CDCl₃) δ 7.54-7.49 (m, 2H), 7.43-7.39 (m, 3H), 7.26-7.22 (m, 2H), 7.16-7.12 (m, 1H), 6.85-6.82 (m, 1H), 3.66-3.57 (m, 1H), 2.53-2.22 (m, 2H), 1.91 (s, 1H), 1.54 (s, 3H).

¹³C NMR (150 MHz, CDCl₃) δ 180.4, 143.8, 134.6, 131.6, 129.8, 128.6, 128.3, 126.8, 124.8 (q, J=282.2 Hz), 123.4, 122.9, 110.0, 68.7 (q, J=31.4 Hz), 46.1, 37.8, 25.8.

¹⁹F NMR (375 MHz, CDCl₃) δ −80.0 (d, J=7.2 Hz, 3F).

IR (ATR): 3377, 3056, 2967, 2926, 1703, 1610, 1506, 1379, 1282, 1163, 1126, 1028, 854, 760 cm⁻¹.

HRMS (ESI, m/z): a theoretically calculated value: C₁₈H₁₆F₃NO₂Na⁺ (M+Na)⁺: 358.1025; found: 358.1012.

### Embodiment 22

### (E)-1,1,1-trifluoro-4-(2-fluorophenyl)-3-ene-butan-2-ol

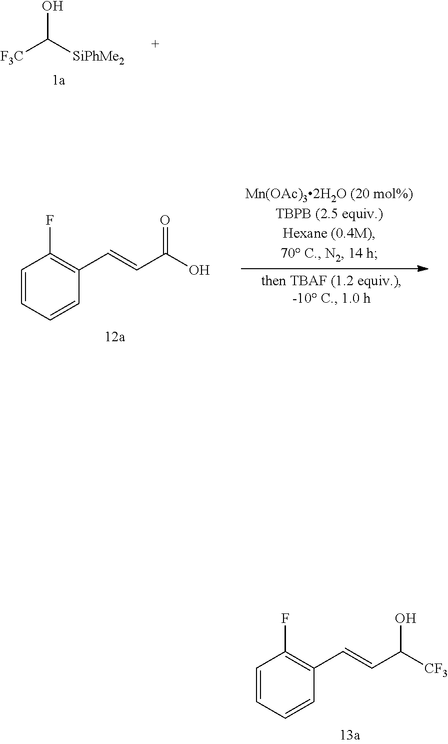

Under a nitrogen atmosphere, a magnetic stir bar was placed into a dry 10 mL Schlenk tube and Mn(OAc)₃.2H₂O (21.4 mg, 0.08 mmol, 20 mol %), 12a (132.8 mg, 0.8 mmol, 2.0 eq.), n-hexane (1 mL, 0.4 M), 1a (93.6 mg, 0.4 mmol) and TBPB (194.3 mg, 1.0 mmol, 2.5 eq.) were added. Subsequently, the tube was sealed, and the mixture was heated to 70° C. and stirred for 18 h. After that, the Schlenk tube was placed in a low temperature tank of −10° C., and TBAF was added to be stirred for 1.0 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1, v/v) to give 64 mg (yield 73%) of the target compound as a white solid. The product was tested, with the results as follows:

R_{f}=0.56 (PE/EA=4/1, v/v).

NMR spectrum:

¹H NMR (400 MHz, CDCl₃) δ 7.47 (t, J=7.2 Hz, 1H), 7.31-7.26 (m, 1H), 7.15 7.00 (m, 3n), 6.31 (dd, J=16.2, 6.4 Hz, 1H), 4.69-4.63 (m, 1H), 2.44 (s, 1H);

¹³C NMR (100 MHz, CDCl₃) δ 160.7 (d, J=250.5 Hz), 130.3 (d, J=8.7 Hz), 129.0 (d, J=2.9 Hz), 128.1 (d, J=2.9 Hz), 124.4 (q, J=273.4 Hz), 124.4 (d, J=3.9 Hz), 123.4, 123.4 (d, J=6.7 Hz), 116.1 (d, J=22.2 Hz), 71.9 (q, J=32.1 Hz);

¹⁹F NMR (375 MHz, CDCl₃) δ −78.9 (d, J=6.0 Hz, 3F), −117.0-−117.0 (m, 1F).

IR (ATR): 3396, 2922, 1659, 1491, 1457, 1267, 1174, 1125, 969, 883, 753 cm⁻¹.

HRMS (ESI, m/z): a theoretically calculated value: C₁₀H₆F₄O⁻ (M H)⁻: 219.0439; found: 219.0441.

### Embodiment 23

### 5,5-difluoro-1-phenyl-4-hydroxy-2-methylenepentan-1-one

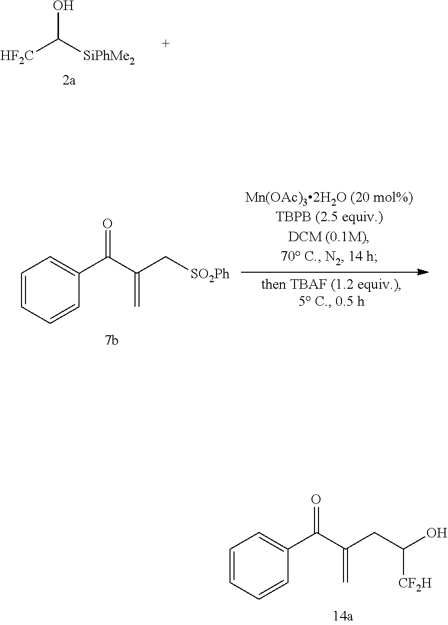

Under a nitrogen protection, Mn(OAc)₃.2H₂O (16.1 mg, 0.06 mmol, 20 mol %) and 7b (257.4 mg, 0.9 mmol, 3.0 eq.) were added into a dry 10 mL Schlenk reaction tube containing a polytetrafluoroethylene magnetic stir bar with a suitable size, followed by addition of DCM (3 mL, 0.1 M), 2a (64.8 mg, 0.3 mmol) and TBPB (145.7 mg, 0.75 mmol, 2.5 eq.). The reaction tube was sealed, and then placed on a heating module and the mixture was heated to 70° C. to be subjected to a reaction for 14 h. The reaction tube was taken down. After cooled to room temperature, the reaction tube was put into an ice water bath, and opened, and TBAF (1.0 M in THF, 0.36 mL, 0.36 mmol, 1.2 eq.) was added. The reaction tube was then sealed and placed in the ice water bath to be stirred for 30 min. The reaction was quenched with 2 mL of water, and the reaction solution was extracted with DCM (3×10 mL). The organic phases were mixed, washed with saturated sodium chloride (2×25 mL), dried with anhydrous Na₂SO₄, filtered, and concentrated with a rotary evaporator under reduced pressure to obtain a crude product. The resulting crude product was separated by silica gel column chromatography with petroleum ether and ethyl acetate as an eluent to obtain 5,5-difluoro-1-phenyl-4-hydroxy-2-methylenepentan-1-one as a colorless liquid (yield 80%). The product was tested, with the results as follows:

R_{f}=0.25 (PE/EA=10/1, v/v).

NMR spectrum:

¹H NMR (400 MHz, CDCl₃) δ 7.77 (d, J=7.3 Hz, 2H), 7.58 (t, J=7.3 Hz, 1H), 7.45 (t, J=7.6 Hz, 2H), 6.11 (s, 1H), 5.86-5.58 (m, 2H), 3.98-3.91 (m, 1H), 3.83 (s, 1H), 2.84-2.63 (m, 2H);

¹³C NMR (100 MHz, CDCl₃) δ 199.5, 143.1, 137.0, 133.0, 130.9, 130.0, 128.5, 116.1 (t, J=244.2 Hz), 70.9 (t, J=24.1 Hz), 33.7;

¹⁹F NMR (375 MHz, CDCl₃) δ −128.5-−131.2 (m, 2F).

IR (ATR): 3452, 3064, 2941, 2292, 2251, 1655, 1446, 1409, 1375, 1330, 1219, 1174, 1140, 1059, 947, 757 cm⁻¹.

HRMS (ESI, m/z): a theoretically calculated value: C₁₂H₁₃F₂O₂⁺ (M+H)⁺: 227.0878; found: 227.0871.

The method for preparing a difluoroethanolation reagent 2a used in the embodiment is as follows:

1) 30 mmol of a compound 4 (the same as the compound 4 in Embodiment 1) and a solvent THF were added to a single-neck flask with a magnetic stirrer and the single-neck flask was placed in an ice bath of 0° C. Concentrated hydrochloric acid (10 eq.) was added dropwise to the reaction solution. After completion of adding dropwise, the reaction device was moved to a condition under room temperature and stirring was continued to be performed. After the reaction was completed, water was added to quench the reaction, followed by separation by column chromatography to obtain a compound 6.

2) 10 mmol of the compound 6 and a solvent methanol (0.2 M) were added to a single-neck flask with a magnetic stirrer and the single-neck flask was placed in an ice bath of 0° C. Sodium borohydride solid (1.1 eq.) was added to the reaction system in three batches. After the reaction was completed, water was added to quench the reaction, followed by separation by column chromatography to obtain the difluoroethanolation reagent 2a.

NMR spectrum of the difluoroethanolation reagent 2a:

¹H NMR (400 MHz, CDCl3) δ 7.57 (d, J=6.4 Hz, 2H), 7.45-7.39 (m, 3H), 5.39 (t, J=54.9 Hz, 1H), 0.62 (s, 6H);

¹³C NMR (100 MHz, CDCl3) δ 233.0 (t, J=32.0 Hz), 134.3, 132.4, 130.5, 128.4, 112.2 (t, J=249.9 Hz), −4.7;

¹⁹F NMR (375 MHz, CDCl3) δ −125.3 (d, J=53.6 Hz, 3F).

IR (ATR): 3071, 2960, 1670, 1428, 1252, 1118, 1044, 828, 787, 697 cm⁻¹.

HRMS (ESI, m/z): a theoretically calculated value: C₁₀H₃F₂OSi⁺ (M+H)⁺: 215.0698; found: 215.0693.

### Embodiment 24

Fluorinated Derivative of Diosgenin

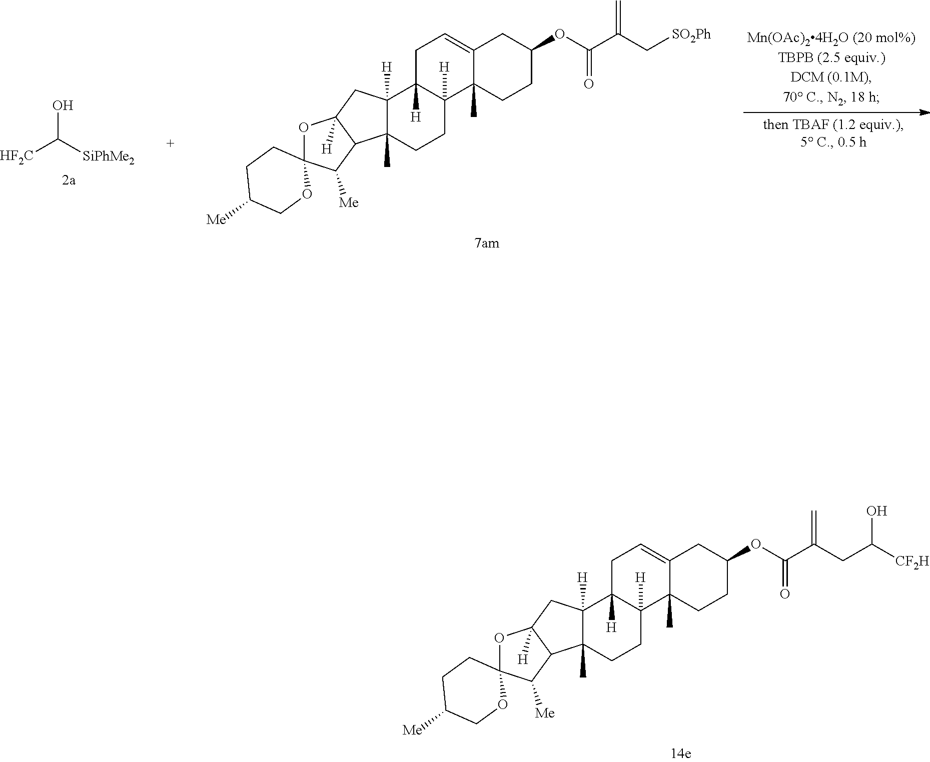

Under a nitrogen protection, Mn(OAc)₂.4H₂O (14.7 mg, 0.06 mmol, 20 mol %) and 7am (373.5 mg, 0.6 mmol, 2.0 eq.) were added into a dry 10 mL Schlenk reaction tube containing a polytetrafluoroethylene magnetic stir bar with a suitable size, followed by addition of DCM (3 mL, 0.1 M), 2a (64.8 mg, 0.3 mmol) and TBPB (145.7 mg, 0.75 mmol, 2.5 eq.). The reaction tube was sealed and then placed on a heating module and the mixture was heated to 70° C. to be subjected to a reaction for 18 h. The reaction tube was taken down, allowed to cool to room temperature, placed in an ice water bath, and opened and then TBAF (1.0 M in THF, 0.36 mL, 0.36 mmol, 1.2 eq.) was added. The reaction tube was then sealed, and placed in an ice water bath to be stirred for 30 min. The reaction was then quenched with 2 mL of water, and the reaction solution was subjected to extraction with DCM (3×10 mL). The organic phases were mixed, then washed with saturated sodium chloride (2×25 mL), dried with anhydrous Na₂SO₄, filtered, and concentrated with a rotary evaporator under reduced pressure to obtain a crude product. The resulting crude product was separated by silica gel column chromatography with petroleum ether and ethyl acetate as an eluent to obtain the difluorinated derivative of diosgenin (yield 67%) as a white solid. The product was tested, with the results as follows:

R_{f}=0.42 (PE/EA=5/1, v/v). mp: 126.1-127.4° C.

NMR spectrum:

¹H NMR (400 MHz, CDCl₃) δ 6.29 (d, J=1.3 Hz, 1H), 5.84-5.52 (m, 2H), 5.39 (d, J=5.1 Hz, 1H), 4.74-4.61 (m, 1H), 4.40 (dd, J=14.3, 8.1 Hz, 1H), 3.98-3.84 (m, 1H), 3.50-3.43 (m, 1H), 3.36 (t, J=10.9 Hz, 1H), 2.69 (dd, J=14.4, 3.3 Hz, 1H), 2.51 (dd, J=14.4, 9.0 Hz, 1H), 2.37 (d, J=7.9 Hz, 2H), 2.06-1.40 (m, 18H), 1.02 (s, 3H), 0.96 (d, J=7.0 Hz, 3H), 0.78 (d, J=4.8 Hz, 6H);

¹³C NMR (100 MHz, CDCl₃) δ 167.4, 139.5, 136.3, 122.8, 116.0 (t, J=244.3 Hz), 109.4, 80.9, 75.3, 70.6 (t, J=23.8 Hz), 67.0, 62.2, 56.6, 41.7, 40.4, 39.8, 38.1, 37.0, 36.9, 33.4 (t, J=4.0 Hz), 32.2, 32.0, 31.5, 28.9, 27.8, 20.9, 19.5, 17.2, 16.4, 14.6;

¹⁹F NMR (375 MHz, CDCl₃) δ −128.7-−131.6 (m, 2F).

IR (ATR): 3418, 2945, 1710, 1454, 1375, 1327, 1245, 1051, 980, 83 cm⁻¹.

HRMS (ESI, m/z): a theoretically calculated value: C₃₃H₄₈F₃O₅⁺ (M+H)⁺: 563.3543; found: 563.3533.

### Embodiment 25

### Methyl 3-(3,3-difluoro-2-hydroxypropyl)-3-methyl-2-oxo-1-phenyldihydroindole-6-carboxylate

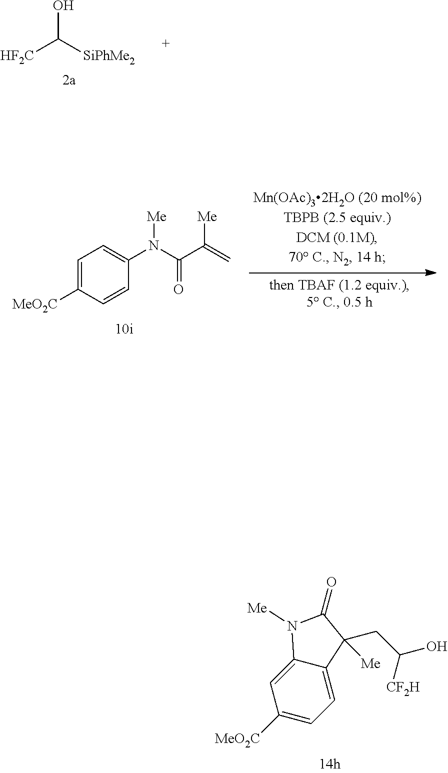

Under a nitrogen protection, Mn(OAc)₃.2H₂O (16.1 mg, 0.06 mmol, 20 mol %) and 10i (83.4 mg, 0.36 mmol, 1.2 eq.) were added into a dry 10 mL Schlenk reaction tube containing a polytetrafluoroethylene magnetic stir bar with a suitable size, followed by addition of DCM (3 mL, 0.1 M), 2a (64.8 mg, 0.3 mmol) and TBPB (145.7 mg, 0.75 mmol, 2.5 eq.). The reaction tube was sealed and then placed on a heating module and the mixture was heated to 70° C. to be subjected to a reaction for 14 h. The reaction tube was taken down, allowed to cool to room temperature, placed in an ice water bath, and opened and TBAF (1.0 M in THF, 0.36 mL, 0.36 mmol, 1.2 eq.) was added. The reaction tube was then sealed, and placed in an ice water bath to be stirred for 30 min. Then the reaction was quenched with 2 mL of water, and the reaction solution was subjected to extraction with DCM (3×10 mL). The organic phases were mixed, washed with saturated sodium chloride and sodium carbonate, dried with anhydrous Na₂SO₄, filtered, and concentrated with a rotary evaporator under reduced pressure to obtain a crude product. The resulting crude product was separated by silica gel column chromatography with petroleum ether and ethyl acetate as an eluent to obtain methyl 3-(3,3-difluoro-2-hydroxypropyl)-3-methyl-2-oxo-1-phenyldihydroindole-6-carboxylate (yield 76%, 14h-a:14h-b=51:49). The product was tested, with the results as follows:

R_{f} (14h-a)=0.28 (PE/EA=2/1, v/v). (36.8 mg, 39% yield, white solid, mp: 107.2-108.9° C.

NMR spectrum of 14h-a:

¹H NMR (400 MHz, CDCl₃) δ 8.05 (dd, J=8.1, 1.7 Hz, 1H), 7.88 (s, 1H), 6.92 (d, J=8.2 Hz, 1H), 5.81-5.52 (m, 1H), 4.44 (s, 1H), 4.19-4.01 (m, 1H), 3.91 (s, 3H), 3.28 (s, 3H), 2.18-1.80 (m, 2H), 1.49 (s, 3H);

¹³C NMR (100 MHz, CDCl₃) δ 182.4, 166.8, 146.4, 134.8, 131.2, 125.5, 123.9, 116.0 (t, J=240.6 Hz), 108.4, 68.6 (t, J=24.1 Hz), 52.3, 46.5, 36.0, 26.9, 22.6;

¹⁹F NMR (375 MHz, CDCl₃) δ −124.8-−133.7 (m, 2F).

IR (ATR): 3414, 2926, 1703, 1498, 1457, 1286, 1103, 1051, 977, 772 cm⁻¹.

HRMS (ESI, m/z): a theoretically calculated value: C₁₅H₁₈F₂NO₄⁺ (M+H)⁺: 314.1198; found: 314.1198.

R_{f} (14h-b)=0.17 (PE/EA=2/1, v/v). (34.5 mg, 37% yield, white solid, mp: 128.4-129.8° C.).

NMR spectrum of 14h-b:

¹H NMR (400 MHz, CDCl₃) δ 8.05 (dd, J=8.1, 1.7 Hz, 1H), 6.92 (d, J=8.2 Hz, 1H), 5.81-5.52 (m, 1H), 4.43 (s, 1H), 4.21-4.07 (m, 1H), 3.91 (s, 3H), 3.27 (s, 3H), 2.23-1.76 (m, 1H), 1.49 (s, 3H);

¹³C NMR (100 MHz, CDCl₃) δ 181.6, 167.0, 147.8, 132.5, 131.2, 124.7, 124.0, 115.8 (t, J=242.5 Hz), 108.1, 68.8 (t, J=23.8 Hz), 52.2, 45.9, 37.2, 26.7, 25.2;

¹⁹F NMR (375 MHz, CDCl₃) δ −126.5-−133.0 (m, 2F).

IR (ATR): 3422, 2922, 1707, 1498, 1457, 1372, 1286, 1055, 977, 772 cm⁻¹.

HRMS (ESI, m/z): theoretically calculated: C₁₅H₁₈F₂NO₄⁺ (M+H)⁺: 314.1198; found: 314.1197.

### Embodiment 26

### (E)-4-(2,6-difluorophenyl)-1,1-difluorobut-3-en-2-ol

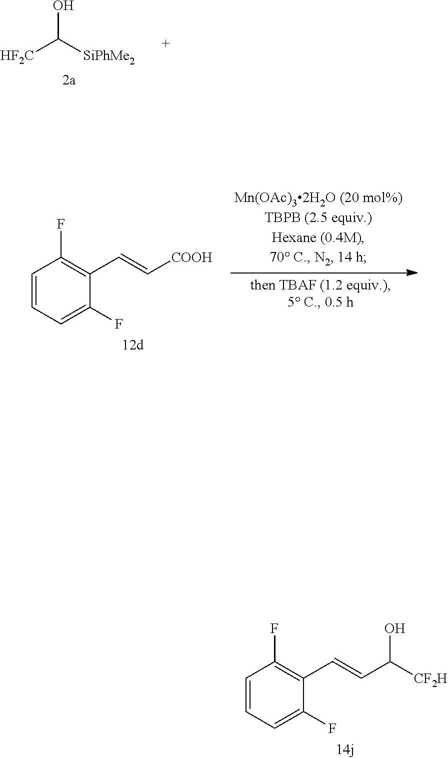

Under a nitrogen protection, Mn(OAc)₃.2H₂O (16.1 mg, 0.06 mmol, 20 mol %) and 12d (110.4 mg, 0.6 mmol, 2.0 eq.) were added into a dry 10 mL Schlenk reaction tube containing a polytetrafluoroethylene magnetic stir bar with a suitable size, followed by addition of DCM (3 mL, 0.1 M), 2a (64.8 mg, 0.3 mmol) and TBPB (145.7 mg, 0.75 mmol, 2.5 eq.). The reaction tube was sealed and then placed on a heating module and the mixture was heated to 70° C. to be subjected to a reaction for 14 h. The reaction tube was taken down, allowed to cool to room temperature, placed in an ice water bath, and opened and TBAF (1.0 M in THF, 0.36 mL, 0.36 mmol, 1.2 eq.) was added. The reaction tube was then sealed and placed in an ice water bath to be stirred for 30 min. The reaction was then quenched with 2 mL of water, and the reaction solution was subjected to extraction with DCM (3×10 mL). The organic phases were mixed, washed with saturated sodium chloride and sodium carbonate, dried with anhydrous Na₂SO₄, filtered, and concentrated with a rotary evaporator under reduced pressure to obtain a crude product. The resulting crude product was separated by silica gel column chromatography with petroleum ether and ethyl acetate as an eluent to obtain (E)-4-(2,6-difluorophenyl)-1,1-difluorobut-3-en-2-ol (yield 60%) as a pale yellow liquid. The product was tested, with the results as follows:

R_{f}=0.40 (PE/EA=5/1, v/v).

NMR spectrum:

¹H NMR (400 MHz, CDCl₃) δ 7.24-7.16 (m, 1H), 6.94-6.84 (m, 3H), 6.59-6.53 (m, 1H), 5.89-5.59 (m, 1H), 4.48 (dq, J=10.3, 5.2 Hz, 1H), 2.23 (s, 1H);

¹³C NMR (150 MHz, CDCl₃) δ 161.2 (dd, J=250.3, 7.3 Hz), 129.7-129.4 (m), 129.2 (t, J=10.8 Hz), 121.2, 115.5 (t, J=243.8 Hz), 113.4 (t, J=15.1 Hz), 111.7 (dd, J=21.5, 4.8 Hz), 72.8 (t, J=24.4 Hz);

¹⁹F NMR (375 MHz, CDCl₃) δ −112.7 (s, 2F), −126.3-−130.0 (m, 2F).

IR (ATR): 3396, 2926, 1621, 1584, 1464, 1267, 1118, 1062, 999, 909 cm⁻¹

HRMS (ESI, m/z): a theoretical value: C₁₀H₈F₄ONa⁺ (M+Na)⁺: 243.0404; found: 243.0412.

### Embodiment 27

### 5,5,5-trifluoro-4-hydroxy-2-methylenepentanoic acid-5-formylpentyl Ester

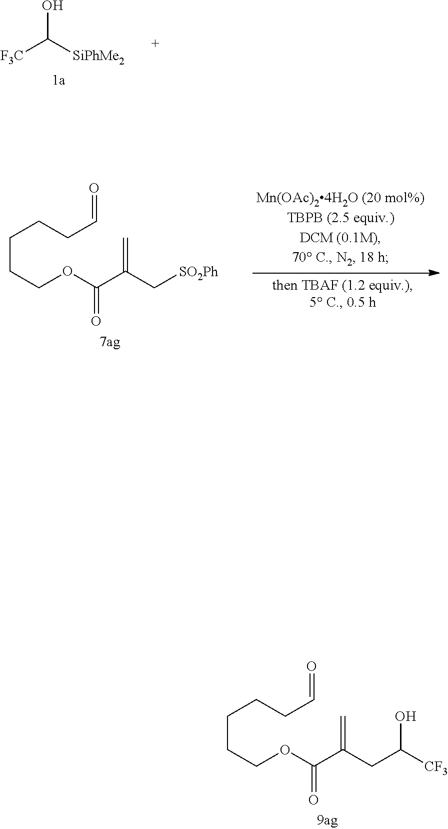

Under a nitrogen atmosphere, a magnetic stir bar was placed into a dry 10 mL Schlenk tube and Mn(OAc)₂.4H₂O (14.7 mg, 0.06 mmol, 20 mol %), 7a (152.4 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice-water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) to give 39 mg (yield 46%) of the target compound as a colorless oil. The product was tested, with the results as follows:

R_{f}=0.61 (PE/EA=2/1, v/v).

NMR spectrum:

¹H NMR (400 MHz, CDCl₃) δ 9.77 (t, J=1.5 Hz, 1H), 6.33 (s, 1H), 5.80 (s, 1H), 4.22-4.09 (m, 3H), 2.79-2.57 (m, 2H), 2.47 (td, J=7.2, 1.3 Hz, 2H), 1.76-1.64 (m, 4H), 1.46-1.38 (m, 2H);

¹³C NMR (100 MHz, CDCl₃) δ 202.5, 167.9, 135.2, 129.8, 124.9 (q, J=281.1 Hz), 69.9 (q, J=30.7 Hz), 65.3, 43.8, 33.5, 28.4, 25.6, 21.7;

¹⁹F NMR (375 MHz, CDCl₃) δ −79.6 (d, J=6.0 Hz, 3F).

IR (ATR): 3425, 2930, 2859, 1711, 1275, 1167, 1126, 1029, 734 cm⁻¹.

HRMS (ESI, m/z): a theoretically calculated value: C₁₂H₁₇F₃NaO₄⁺ (M+Na)⁺: 305.0971; found: 305.0970.

### Embodiment 28

Cholesterol Derivative

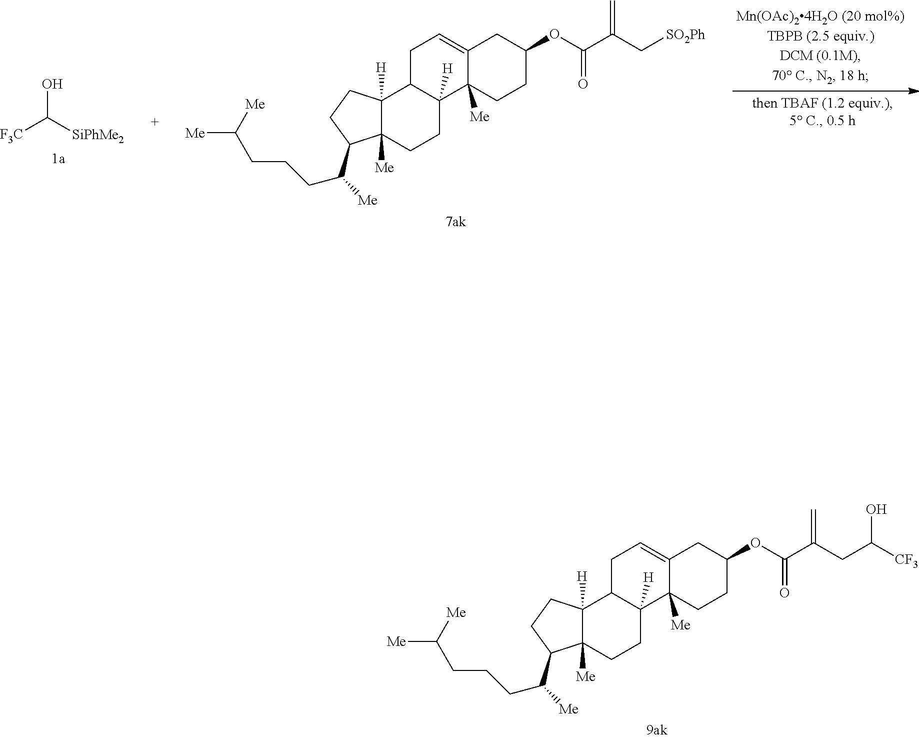

Under a nitrogen atmosphere, a magnetic stir bar was placed into a dry 10 mL Schlenk tube and Mn(OAc)₂.4H₂O (14.7 mg, 0.06 mmol, 20 mol %), 7ak (357.6 mg, 0.6 mmol, 2.0 eq.), DCM (3 mL, 0.1 M), 1a (70.2 mg, 0.3 mmol) and TBPB (145.7 mg, 0.75 mmol, 2.5 eq.) were added. Subsequently, the tube was sealed, and the mixture was heated to 70° C. and stirred for 18 h. After that, the Schlenk tube was placed in an ice water bath of 5° C., and TBAF was added to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (5/1, v/v) to give 53 mg (yield 71%) of the target compound as a yellow oil. The product was tested, with the results as follows:

R_{f}=0.48 (PE/EA=8/1, v/v). mp: 83-85° C.

NMR spectrum:

¹H NMR (400 MHz, CDCl₃) δ 6.33 (s, 1H), 5.78 (s, 1H), 5.40 (d, J=4.3 Hz, 1H), 4.73-4.65 (m, 1H), 4.12-4.09 (m, 1H), 3.75 (s, 1H), 2.77-2.57 (m, 2H), 2.37 (d, J=7.6 Hz, 2H), 2.03-1.95 (m, 2H), 1.92-1.79 (m, 3H), 1.70-1.43 (m, 7H), 1.40-1.25 (m, 4H), 1.20-1.08 (m, 7H), 1.03-0.95 (m, 6H), 0.91 (d, J=6.4 Hz, 3H), 0.86 (dd, J=6.7, 1.8 Hz, 6H), 0.68 (s, 3H);

¹³C NMR (100 MHz, CDCl₃) δ 167.6, 139.4, 135.6, 129.6, 124.9 (q, J=280.8 Hz), 123.2, 75.64, 70.1 (q, J=30.7 Hz), 56.8, 56.2, 50.1, 42.4, 39.8, 39.6, 38.1, 37.0, 36.7, 36.3, 35.9, 33.6, 32.0, 31.9, 28.4, 28.1, 27.8, 24.4, 24.0, 23.0, 22.7, 21.2, 19.5, 18.8, 12.0;

¹⁹F NMR (375 MHz, CDCl₃) δ −79.5 (bs, 3F).

IR (ATR): 3422, 2937, 2870, 1711, 1633, 1465, 1331, 1275, 1170, 1129, 1029, 734 cm⁻¹.

HRMS (ESI, m/z): a theoretically calculated value: C₃₃H₅₁F₃NaO₃⁺ (M+Na)⁺: 575.3683; found: 575.3671.

### Embodiment 29

A method for synthesizing a compound L (1,1,1-trifluoro-3-ene-4-(2-methyl-3-(3-(3,4-dihydroxymethyl)phenyl)propyl)phenylbutan-2-ol) with anticancer activity and its difluorinated analogue M (1,1-difluoro-3-ene-4-(2-methyl-3-(3-(3,4-dihydroxymethyl)phenyl)propyl)phenylbutan-2-ol) based on the trifluoroethanolation reagent 1a or the difluoroethanolation reagent 2a. The reaction equation of the synthetic method may be expressed as follows:

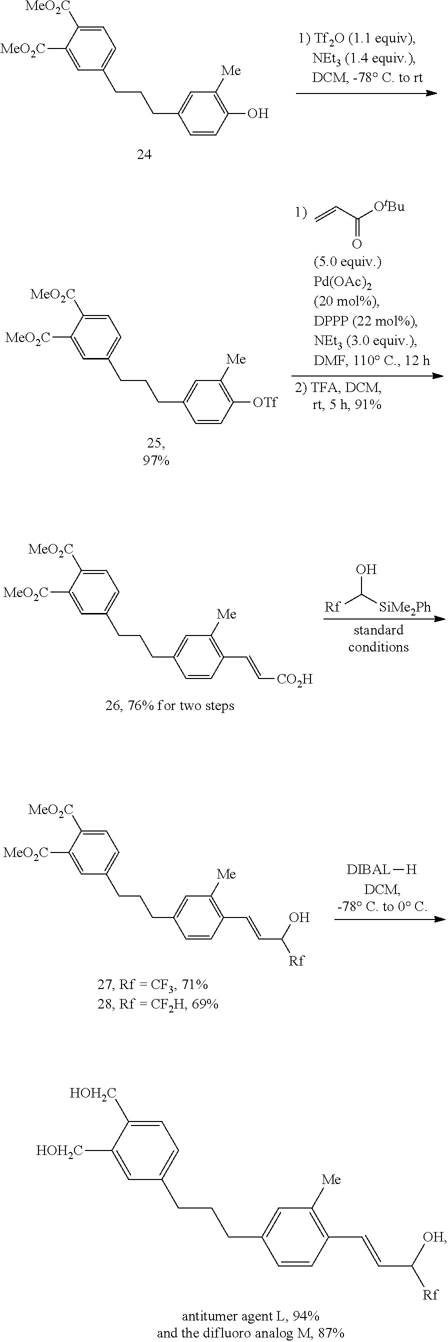

wherein R_{f} is CF₃— or HCF₂—, and when R_{f} is CF₃—, the resulting product is the compound L with anticancer activity, and when R_{f} is HCF₂—, the resulting product is the difluorinated analogue M of the compound L.

Specifically, the synthetic method includes the following steps:

1) A reactant 24 and triethylamine (1.4 eq.) were dissolved in a solvent DCM under nitrogen protection, and then the reaction was placed in a low temperature bath of −78° C., and trifluoromethanesulfonic anhydride (1.1 eq.) was added slowly dropwise. After completion of the adding dropwise, the reaction was quenched with a saturated aqueous ammonium chloride solution, and extraction was performed by DCM, followed by separation by column chromatography to obtain a compound 25;

2) the compound 25 and tert-butyl acrylate (5 eq.), in the reaction system of palladium acetate (20% of the molar amount of the compound 25) and DPPP (1,3-bis(diphenylphosphino)propane, 22% of the molar amount of the compound 25) as a catalytic system, triethylamine (3.0 eq.) as a base, and DMF as a solvent, were stirred at 110° C. for 12 h. Water was added to quench the reaction, followed by extraction with ethyl acetate. The compound obtained by column chromatography was treated with trifluoroacetic acid in a solvent dichloromethane to obtain a compound 26;

3) by using the reaction of the compound 26 with the trifluoroethanolation reagent 1a or the difluoroethanolation reagent 2a, Mn(OAc)₃.2H₂O (16.1 mg, 0.06 mmol, 20 mol %) and 26 (0.6 mmol, 2.0 eq.) were added into a dry 10 mL Schlenk reaction tube containing a polytetrafluoroethylene magnetic stir bar with a suitable size, followed by addition of DCM (3 mL, 0.1 M), 1a or 2a (0.3 mmol) and TBPB (145.7 mg, 0.75 mmol, 2.5 eq.). The reaction tube was sealed, and then placed on a heating module and the mixture was heated to 70° C. to be subjected to a reaction for 14 h. The reaction tube was taken down and allowed to cool to room temperature, and then placed in an ice water bath, and opened and TBAF (1.0 M in THF, 0.36 mL, 0.36 mmol, 1.2 eq.) was added. The reaction tube was then sealed and placed in an ice water bath to be stirred for 30 min. The reaction was then quenched with 2 mL of water, and extraction was performed with DCM (3×10 mL), followed by separation by column chromatography to obtain the target product 27 or compound 28; and

(4) the compound 27 or compound 28 was dissolved in a dichloromethane solvent, and the reaction was placed in a low temperature tank of −78° C., and DIBAL-H (diisobutylaluminium hydride) was added slowly dropwise. After completion of adding dropwise, the reaction solution was slowly heated to 0° C., and the reaction was quenched with 3M HCl, followed by separation by column chromatography to obtain the compound L with anticancer activity or the difluorinated analogue M.

### Embodiment 30

Ethyl 5,5,5-trifluoro-4-hydroxy-2-methylene pentanoate, with a synthetic route and a preparation method as follows:

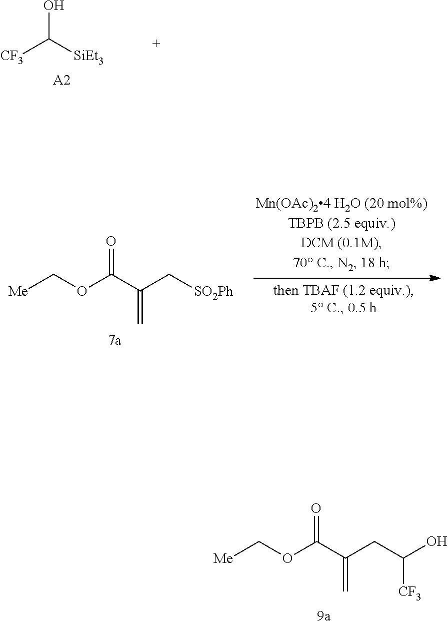

Under a nitrogen atmosphere, a magnetic stir bar was placed into a dry 10 mL Schlenk tube and Mn(OAc)₂.4H₂O (14.7 mg, 0.06 mmol, 20 mol %), 7a (152.4 mg, 0.6 mmol, 2.0 eq.), DCM (3 mL, 0.1 M), A2 (0.3 mmol) and TBPB (145.7 mg, 0.75 mmol, 2.5 eq.) were added. Subsequently, the tube was sealed, and the mixture was heated to 70° C. and stirred for 18 h. After that, TBAF was added to the stirred material in an ice-water bath of 5° C. to be stirred for 0.5 h. The reaction mixture was quenched with water (2 mL) and subjected to extraction with DCM (3×10 mL), the organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) (PE: petroleum ether, EA: ethyl acetate) to give 35 mg (yield 47%).

The preparation method of the trifluoroethanolation reagent A2 used in the embodiment was as follows:

1) triethylchlorosilane (30 mmol), trifluoroethanol (30 mmol) and a tetrahydrofuran solvent were added to a dry single-neck flask with a magnetic stirrer, and the reaction flask was placed in a low-temperature tank of −78° C., and LDA (lithium diisopropylamide, 105 mmol) was added dropwise with a syringe pump. After completion of adding dropwise, the mixture was kept to be stirred for 4 h, heated to room temperature, and stirred again until the trifluoroethanol was completely consumed. Triethylchlorosilane was added at 0° C. to be stirred for 4 h, followed by separation by column chromatography to obtain a compound 4 (1,1-difluoro-2-triethylsilyl-2-triethylsiloxyethylene);

2) a fluorinating agent Select-Fluor (2.0 eq.) and a mixed solvent of acetonitrile and dichloromethane (4:1, v/v) were added to a dry single-neck flask with a magnetic stirrer, and the single-neck flask was placed in an ice-water bath of 0° C. The compound 4 was added to the reaction, and after completion, the reaction solution was stirred at room temperature for 12 h. The reaction was quenched with water, followed by separation by column chromatography to obtain a compound 5 (trifluoroacetyltriethylsilane); and

3) the compound 5 and methanol were added to a single-neck flask, and sodium borohydride solid was added to the reaction system in three batches. After the reaction was completed, water was added to quench the reaction, followed by separation by column chromatography to obtain the trifluoroethanolation reagent A2.

### Embodiment 31

Ethyl 5,5,5-trifluoro-4-hydroxy-2-methylene pentanoate, using A3 as a raw material, with a synthetic route and a preparation method as follows:

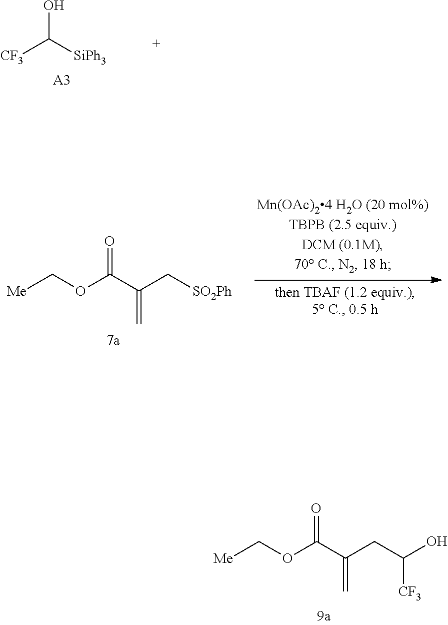

Under a nitrogen atmosphere, a magnetic stir bar was placed into a dry 10 mL Schlenk tube, and Mn(OAc)₂.4H₂O (14.7 mg, 0.06 mmol, 20 mol %), 7a (152.4 mg, 0.6 mmol, 2.0 eq.), DCM (3 mL, 0.1 M), A3 (0.3 mmol) and TBPB (145.7 mg, 0.75 mmol, 2.5 eq.) were added. Subsequently, the tube was sealed, and the mixture was heated to 70° C. and stirred for 18 h. After that, TBAF was added to the stirred material in an ice-water bath of 5° C. to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) (PE: petroleum ether, EA: ethyl acetate) to give 25.4 mg (yield 34%).

The preparation method of the trifluoroethanolation reagent A3 used in the embodiment was as follows:

1) triphenylsilyl chloride (30 mmol), trifluoroethanol (30 mmol), HMPA (3 mL) and a tetrahydrofuran solvent were added to a dry single-neck flask with a magnetic stirrer, and the reaction flask was placed in a low-temperature tank of −78° C., and LDA (lithium diisopropylamide, 105 mmol) was added dropwise with a syringe pump. After completion of adding dropwise, the mixture was kept to be stirred for 4 h, heated to room temperature, and then stirred again until trifluoroethanol was completely consumed. Triethylchlorosilane was added at 0° C. to be stirred for 4 h, followed by separation by column chromatography to obtain a compound 4 (1,1-difluoro-2-triphenylsilyl-2-triethylsiloxyethylene);

2) a fluorinating agent Select-Fluor (2.0 eq.) and a mixed solvent of acetonitrile and dichloromethane (4:1, v/v) were added to a dry single-neck flask with a magnetic stirrer, and the single-neck flask was placed in an ice-water bath of 0° C. The compound 4 was added to the reaction, and after completion, the reaction solution was stirred at room temperature for 12 h. The reaction was quenched with water, followed by separation by column chromatography to obtain a compound 5 (trifluoroacetyltriphenylsilane); and

3) the Compound 5 and methanol were added to a single-neck flask, and sodium borohydride solid was added to the reaction system in three batches. After the reaction was completed, water was added to quench the reaction, followed by separation by column chromatography to obtain the trifluoroethanolation reagent A3.

### Embodiment 32

Ethyl 5,5,5-trifluoro-4-hydroxy-2-methylene pentanoate, using A4 as a raw material, with a synthetic route and a preparation method as follows:

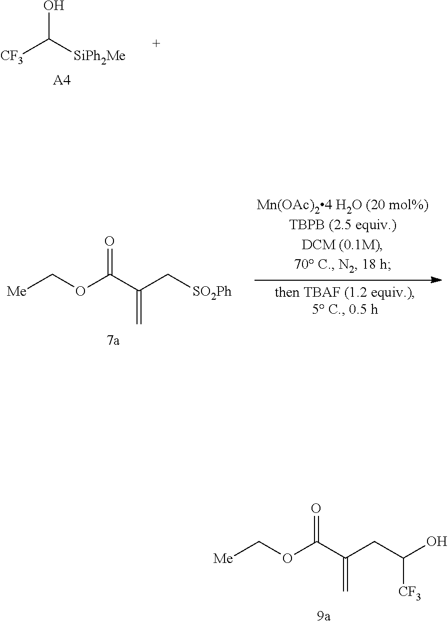

Under a nitrogen atmosphere, a magnetic stir bar was placed into a dry 10 mL Schlenk tube and Mn(OAc)₂.4H₂O (14.7 mg, 0.06 mmol, 20 mol %), 7a (152.4 mg, 0.6 mmol, 2.0 eq.), DCM (3 mL, 0.1 M), A4 (0.3 mmol) and TBPB (145.7 mg, 0.75 mmol, 2.5 eq.) were added. Subsequently, the tube was sealed, and the mixture was heated to 70° C. and stirred for 18 h. After that, TBAF was added to the stirred material in an ice-water bath of 5° C. to be stirred for 0.5 h. The reaction mixture was quenched with water (2 mL) and subjected to extraction with DCM (3×10 mL). The organic phases were mixed and washed with brine, dried over Na₂SO₄, and evaporated to dryness by using a rotary evaporator. The crude product was purified by silica gel column chromatography (200×300-mesh) and eluted with PE/EA (20/1-10/1, v/v) (PE: petroleum ether, EA: ethyl acetate) to give 35.8 mg (yield 48%).

The preparation method of the trifluoroethanolation reagent A4 used in the embodiment was as follows:

1) triphenylsilyl chloride (30 mmol), trifluoroethanol (30 mmol), HMPA (3 mL) and a tetrahydrofuran solvent were added to a dry single-neck flask with a magnetic stirrer, and the reaction flask was placed in a low-temperature tank of −78° C., and LDA (lithium diisopropylamide, 105 mmol) was added dropwise with a syringe pump. After completion of adding dropwise, the mixture was kept to be stirred for 4 h, heated to room temperature, and then stirred again until trifluoroethanol was completely consumed. Triethylchlorosilane was added at 0° C. to be stirred for 4 h, followed by separation by column chromatography to obtain a compound 4 (1,1-difluoro-2-methyldiphenylsilyl-2-triethylsiloxyethylene);

2) a fluorinating agent Select-Fluor (2.0 eq.) and a mixed solvent of acetonitrile and dichloromethane (4:1, v/v) were added to a dry single-neck flask with a magnetic stirrer, and the single-neck flask was placed in an ice-water bath of 0° C. The compound 4 was added to the reaction, and after completion, the reaction solution was stirred at room temperature for 12 h. The reaction was quenched with water, followed by separation by column chromatography to obtain a compound 5 (trifluoroacetylmethyldiphenylsilane); and

3) the compound 5 and methanol were added to a single-neck flask, and sodium borohydride solid was added to the reaction system in three batches. After the reaction was completed, water was added to quench the reaction, followed by separation by column chromatography to obtain the trifluoroethanolation reagent A4.

### Embodiment 33

The storage stability, light stability and solubility in common solvents of the tri(di)fluoroethanolation reagents 1a, 2a, A2, A3 and A4 prepared in Embodiment 1, Embodiment 23, Embodiment 30, Embodiment 31 and Embodiment 32 of the present disclosure were tested, and the results are shown in Table 1 below.

TABLE 1

| | | | | | storage stability | | light stability |
|---|---|---|---|---|---|---|---|
| | | | | | | sealed | continuous |
| | | | | | sealed | storage at | white light |
| | | | | | storage at | 5° C. for | irradiation at |
| | | | | | 30° C. for | two | 25° C. for 2 |
| | solubility | | | | two weeks | weeks | days |
| reagent sample | dichloromethane (DCM) | ethyl acetate (EA) | acetonitrile | n-hexane | (degradation rate, mol %) | | |
| a1 | dissolved | dissolved | dissolved | dissolved | 5% | <1% | <1% |
| a2 | dissolved | dissolved | dissolved | dissolved | 4% | <1% | <1% |
| A2 | dissolved | dissolved | dissolved | dissolved | 14% | 2% | <1% |
| A3 | dissolved | dissolved | dissolved | slightly | <1% | <1% | <1% |
| | | | | soluble | | | |
| A4 | dissolved | dissolved | dissolved | well | 3.2% | <1% | <1% |

What is claimed is:

1. A compound, having a chemical structure formula of: 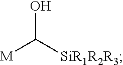
wherein M is selected from a group consisting of CF₃ or CF₂H, R₁ is selected from a group consisting of aryl, heteroaryl, and alkyl, R₂ is selected from a group consisting of aryl, heteroaryl, and alkyl, and R₃ is selected from a group consisting of aryl, heteroaryl, and alkyl.

2. The compound according to claim 1, wherein, the aryl is selected from a group consisting of phenyl, biphenyl, naphthyl, terphenyl, anthracenyl, and substituted aryl.

3. The compound according to claim 1, wherein, the heteroaryl is selected from a group consisting of pyridyl, pyrimidinyl, pyrazinyl, pyridazinyl, triazinyl, furanyl, thienyl, imidazolyl, triazolyl, tetrazolyl, thiazolyl, isothiazolyl, 1,2,4-thiadiazolyl, pyrrolyl, pyrazolyl, oxazolyl, isoxazolyl, oxadiazolyl, benzofuranyl, benzothienyl, benzothiazolyl, indolyl, indazolyl, quinolinyl, isoquinolinyl, purinyl, carbazolyl, benzimidazolyl, pyrrolopyridyl, pyrrolopyrimidinyl, pyrazolopyridyl, pyrazolopyrimidinyl, acridinyl, phenazinyl, benzoxazolyl, benzothiadiazolyl, benzoxadiazolyl, benzotriazolyl, isoquinolinyl, indyl, isothiazolyl, pseudoindolyl, oxadiazolyl, purinyl, phthalazinyl, pteridyl, quinazolinyl, quinoxalinyl, triazinyl, and thiadiazolyl.

4. The compound according to claim 1, wherein, the alkyl is selected from a group consisting of methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, tert-butyl, n-pentyl, iso-pentyl, neopentyl, tert-pentyl, hexyl, heptyl, and octyl.

5. The compound according to claim 1, wherein, the compound is one of the following substances:

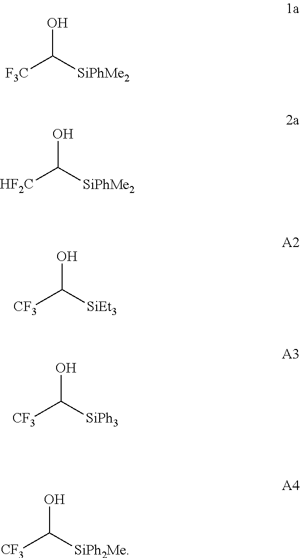

6. A method for preparing the compound according to claim 1, comprising the following steps when M is CF₃:
1) adding R₁, R₂, R₃-trisubstituted chlorosilane, trifluoroethanol, hexamethylphosphoramide into a solvent, performing cooling to −80° C. to −60° C., and then adding dropwise lithium diisopropylamide with a syringe pump; after completion of adding dropwise, performing thorough stirring, then performing heating to room temperature, and performing stirring again until the trifluoroethanol is completely consumed; adding triethylchlorosilane at 0-4° C., carrying out a reaction under stirring, and performing separating by column chromatography to obtain a compound 4;
2) adding a fluorinating agent Select-Fluor into a mixed solvent of acetonitrile and dichloromethane, and adding the compound 4 at 0-4° C., and subsequently carrying out a reaction at room temperature for 8-12 h, quenching the reaction with water, and performing separating by column chromatography to obtain a compound 5; and
3) adding the compound 5 into methanol, followed by addition of sodium borohydride in batches, and carrying out a reaction at 0-4° C.; after completion of the reaction, adding water to quench the reaction, and performing separating by column chromatography to obtain a compound A;
wherein the chemical equation is as follows:

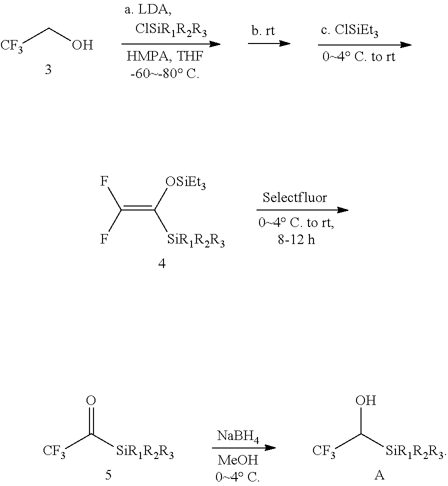

7. A method for preparing the compound according to claim 1, comprising the following steps when M is CF₂H:
1) adding a compound 4 into a solvent, and adding dropwise concentrated hydrochloric acid to the resulting solution at 0-4° C.; after completion of adding dropwise, performing heating to room temperature and carrying out a reaction under stirring; after completion of the reaction, adding water to quench the reaction, and performing separating by column chromatography to obtain a compound 6; and
2) dissolving the compound 6 in a solvent, adding sodium borohydride in batches at 0-4° C., and carrying out a reaction; and after completion of the reaction, adding water to quench the reaction, and performing separating by column chromatography to obtain a compound B;
wherein the chemical equation is as follows:

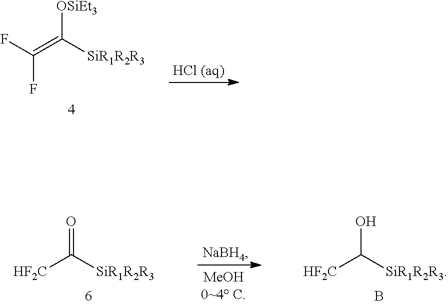

8. An application of the compound according to claim 1, wherein the compound is used as a trifluoroethanolation reagent when M is CF₃, and the compound is used as a difluoroethanolation reagent when M is CF₂H.

9. The application according to claim 8, wherein, the trifluoroethanolation reagent is subjected to allylation to prepare a trifluoromethyl homoallyl alcohol compound, specifically by the steps of: carrying out a reaction between the trifluoroethanolation reagent, allyl sulfone, a catalyst and an oxidant under stirring in an organic solvent at 50-100° C. under nitrogen protection; after completion of the reaction, quenching the reaction by a tetrabutylammonium fluoride solution, and then performing separating and purifying to obtain the corresponding α-trifluoromethyl homoallyl alcohol compound with the following reaction formula: 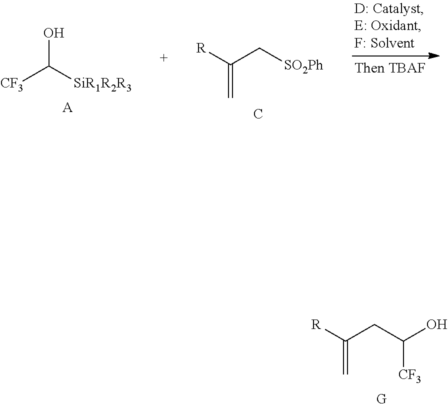
wherein the R group represents a substituent group on allyl sulfone and is aryl, heteroaryl, alkyl, substituted alkyl, aroyl, alkanoyl, substituted oxyacyl, substituted aminoacyl, halogen, sulfonyl, substituted sulfuryl, alkenyl, alkynyl, cyano, nitro, amido or an aldehyde group.

10. The application according to claim 8, wherein, the trifluoroethanolation reagent and acrylamide are subjected to an alkylation reaction to prepare a trifluoromethyl alkyl alcohol compound, specifically by the steps of: carrying out a reaction between the trifluoroethanolation reagent, acrylamide, a catalyst and an oxidant under stirring in an organic solvent at 50-100° C. under nitrogen protection; after completion of the reaction, quenching the reaction by a tetrabutylammonium fluoride solution, and then performing separating and purifying to obtain the corresponding α-trifluoromethyl alkyl alcohol compound I with the following reaction formula: 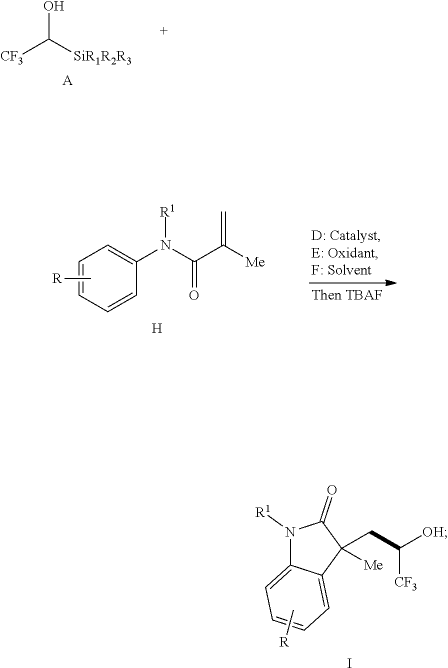
wherein the R group represents a substituent group on the aromatic ring of acrylamide, and includes aryl, alkyl, aroyl, alkanoyl, substituted oxyacyl, halogen, cyano, nitro or alkoxy.

11. The application according to claim 8, wherein, the trifluoroethanolation reagent and cinnamic acid are subjected to an alkenylation reaction to prepare a trifluoromethyl allyl alcohol compound, specifically by the steps of: carrying out a reaction between the trifluoroethanolation reagent, cinnamic acid, a catalyst and an oxidant under stirring in an organic solvent at 50-100° C. under nitrogen protection; after completion of the reaction, quenching the reaction by a tetrabutylammonium fluoride solution, and then performing separating and purifying to obtain the corresponding α-trifluoromethyl allyl alcohol compound with the following reaction formula: 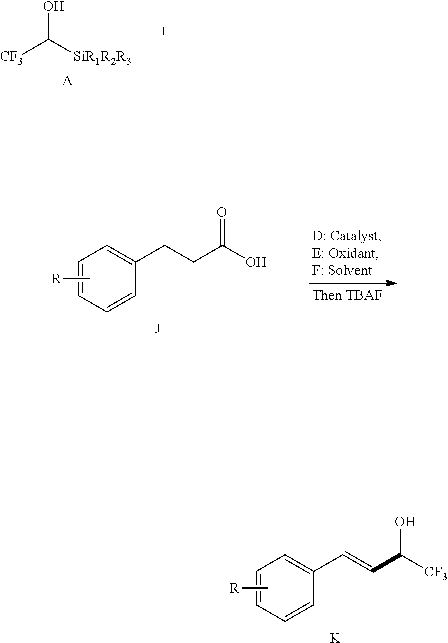
wherein the R group represents a substituent group on the aromatic ring of cinnamic acid, and is selected from a group consisting of aryl, alkyl, aroyl, alkanoyl, substituted oxyacyl, halogen, and alkoxy, with the substituent group being one or more, and the substituent groups being the same or different when a plurality of substituent groups are contained.

12. The application according to claim 8, wherein, the difluoroethanolation reagent and allyl sulfone are subjected to a reaction to prepare a difluoromethyl homoallyl alcohol compound, specifically by the steps of: carrying out a reaction between the difluoroethanolation reagent, allyl sulfone, a catalyst, an oxidant under stirring in an organic solvent at 50-100° C. under nitrogen protection; after completion of the reaction, quenching the reaction by a tetrabutylammonium fluoride solution, and then performing separating and purifying to obtain the α-difluoromethyl homoallyl alcohol compound.

13. The application according to claim 8, wherein, the difluoroethanolation reagent and acrylamide are subjected to a reaction to prepare a difluoromethyl alkyl alcohol compound, specifically by the steps of: carrying out a reaction between the difluoroethanolation reagent, acrylamide, a catalyst, an oxidizer under stirring in an organic solvent at 50-100° C. under nitrogen protection; after completion of the reaction, quenching the reaction by a tetrabutylammonium fluoride solution, and then performing separating and purifying to obtain the α-difluoromethyl alkyl alcohol compound.

14. The application according to claim 8, wherein, the difluoroethanolation reagent and cinnamic acid are subjected to a reaction to prepare a difluoromethyl allyl alcohol compound, specifically by the steps of: carrying out a reaction between the difluoroethanolation reagent, cinnamic acid, a catalyst and an oxidant under stirring in an organic solvent at 50-100° C. under nitrogen protection; after completion of the reaction, quenching the reaction by a tetrabutylammonium fluoride solution, and then performing separating and purifying to obtain the α-difluoromethyl alkyl alcohol compound.
